(19) United States
(12) Patent Application Publication
(10) Pub. No.: US 2025/0009346 A1
(43) Pub. Date: Jan. 9, 2025

(54) Balloon Devices and Methods for Depth-Selective Tissue Biopsy, Stricture Dilation, Mucosal Resurfacing, and Clinical Agent Administration

(71) Applicant: SAINATH INTELLECTUAL PROPERTIES, LLC, Pinellas Park, FL (US)

(72) Inventor: Venkata Sesha Sayi Nath Iyunni, Pinellas Park, FL (US)

(21) Appl. No.: 18/711,023

(22) PCT Filed: Dec. 16, 2021
(86) PCT No.: PCT/US2021/063755

§ 371 Date: May 16, 2024

(51) Int. Cl. A61B 10/02; A61M 25/10

(52) U.S. Cl. CPC A61B 10/02; A61M 25/10; A61B 2010/0225; A61M 2025/1086

(57) ABSTRACT

The invention relates to devices and methods for depth-selective tissue biopsy and segregation, tubular stricture dilation, mucosal ablation and resurfacing, and clinical agent administration using balloon devices, including devices for selective access to different depths of a target tissue.

### FIELD OF THE INVENTION

This present invention relates to devices and methods for tissue biopsy and segregation, tubular stricture dilation, mucosal ablation and resurfacing, and clinical agent administration.

### BACKGROUND

Current tissue biopsy devices do not provide adequate clinical sampling of the different depths or layers of a tissue. The lack of stratified sampling often leads to errors and missed diagnoses. Similarly, devices for the administration of clinical agents (e.g., diagnostic agents, imaging agents, staining agents, visualization agents, detection agents, treatment agents, cytotoxic agents, immunological agents, antimicrobial agents, radioactive agents, tissue-bulking agents, sclerosing agents, acidic agents, timed-release chemical agents, stimulant agents, fibrosis-inducing agents, anti-platelet agents, chemotherapeutic agents, or marking agents, etc., or a combination thereof) do not provide adequate administration to the different depths or layers of a tissue. This lack of stratified administration can lead to errors and poor clinical management.

Current tissue biopsy devices do not adequately contain the tissue sample once it is collected. The lack of containment can lead to lost tissue as well as entry of other biological material from neighboring tissues (i.e., contamination). The tissue loss and tissue contamination often lead to errors and missed diagnoses Similarly, current clinical agent administration devices do not adequately contain the clinical agent prior to administration. The lack of containment can lead to lost clinical agent and decreased efficacy at the target tissue, as well as unintended administration of the clinical agent at neighboring tissues—possibly resulting in potential harm to untargeted tissues. This loss of clinical agent and unintended administration of clinical agent can lead to errors and poor clinical management.

Current techniques for removing strictures using balloon catheter dilation can be limited as they rely on spherical or cylindrical balloons with a uniform diameter, which is typically determined by the diameter of the catheter that encases it. As a result, practitioners must rely on feel when filling balloons in situations where less than total balloon dilation is needed to safely clear a stricture. Errors in estimation in these cases can lead to trauma and perforation when a practitioner inflates too much or inefficient stricture treatment when inflated too little.

Current techniques to foster mucosal resurfacing, such as radiofrequency ablation, cryoablation, laser ablation, and hydrothermal ablation, inadequately protect against the risk of deep tissue burns, perforation, and stricture formation. These techniques may cause unintended trauma to the target site and surrounding regions, leading to an increase in recovery time. Further, these techniques typically require expensive, external equipment and a dedicated power source.

EP2713890B1 describes an expandable device for use in aerodigestive body lumens. Specifically, EP2713890B1 discloses the use of a rounded balloon for sampling of or administration of a clinical agent to an aerodigestive body lumen. EP2713890B1 does not discuss tubular stricture dilation, mucosal ablation and resurfacing, or non-aerodigestive body lumens. Nor does EP2713890B1 disclose a balloon of varying diameters to allow for depth-selective access to different layers of a tissue or the use of a sheath and spheres encasing a balloon to contain material on the balloon.

### SUMMARY OF THE INVENTION

The present invention solves particular problems presented by the prior art related to tissue biopsy, stricture dilation, mucosal resurfacing, and clinical agent administration devices. Specifically, certain embodiments of the invention disclosed herein allow for selective access to different depths of a target tissue and/or precision dilation of a targeted stricture.

Certain embodiments of the invention relate to balloon devices with a balloon of varying diameters useful for tissue biopsy, stricture dilation, mucosal resurfacing, and clinical agent administration.

Certain embodiments of the invention relate to balloon devices with a sheath and stoppers to contain and protect some or all of the material collected on the balloon useful for tissue biopsy and segregation, stricture dilation, mucosal ablation and resurfacing, and clinical agent administration.

Certain embodiments of the invention relate to balloon devices operable for use in conjunction with one or more guidewires useful for tissue biopsy and segregation, stricture dilation, mucosal ablation and resurfacing, and clinical agent administration.

Certain embodiments of the invention include methods for selective biopsy, specifically, collection of tissue from different depths of a target tissue.

Certain embodiments of the invention include methods for selective administration of one or more clinical agents to different depths of a target tissue.

Certain embodiments of the invention include methods for precision dilation of strictures within tubular organs or hollow areas of the body.

Certain embodiments of the invention include methods for mucosal resurfacing within tubular organs or hollow areas of the body-.

### BRIEF DESCRIPTION OF THE DRAWINGS

Other features of the invention disclosed herein will become apparent from the attached drawings, which illustrate embodiments of balloon devices and their component parts, wherein:

FIG. 1A is a schematic view of an embodiment of a balloon device in accordance with the invention:

FIG. 1B is a schematic view of an embodiment of a balloon device in accordance with the invention:

FIG. 2A is a schematic view of an embodiment of a balloon device in accordance with the invention:

FIG. 2B is a schematic view of an embodiment of a balloon device in accordance with the invention:

FIG. 2C is a schematic view of an embodiment of a balloon device in accordance with the invention:

FIG. 2D is a schematic view of an embodiment of a balloon device in accordance with the invention;

FIG. 2E is a schematic view of an embodiment of a balloon device in accordance with the invention;

FIG. 3A is a schematic view of an embodiment of a balloon device in accordance with the invention;

FIG. 3B is a schematic view of an embodiment of a balloon device in accordance with the invention;

FIG. 4A is a schematic view of an embodiment of a balloon device in accordance with the invention;

FIG. 4B is a schematic view of an embodiment of a balloon device in accordance with the invention;

FIG. 5A is a schematic view of an embodiment of a balloon device comprising a sheath and stoppers encasing a balloon in accordance with the invention:

FIG. 5B is a schematic view of an embodiment of a balloon device comprising a sheath and stoppers encasing a balloon in accordance with the invention;

FIG. 5C is a schematic view of an embodiment of a balloon device comprising a sheath and stoppers encasing a balloon in accordance with the invention;

FIG. 5D is a schematic view of an embodiment of a balloon device comprising a sheath and stoppers encasing a balloon in accordance with the invention;

FIG. 5E is a schematic view of an embodiment of a balloon device comprising a sheath and stoppers encasing a balloon in accordance with the invention;

FIG. 5F is a schematic view of an embodiment of a balloon device comprising a sheath and stoppers encasing a balloon in accordance with the invention;

FIG. 6A is a schematic view of an embodiment of a balloon device operable for use in conjunction with one or more guidewires in accordance with the invention;

FIG. 6B is a schematic view of cross-section “6B” of the balloon device of FIG. 6A;

FIG. 6C is a schematic view of detail “6C” of the balloon device of FIG. 6A;

FIG. 7A is a diagram illustrating a method for taking tissue biopsy samples using embodiments of balloon devices in accordance with the invention;

FIG. 7B is a diagram illustrating a method for taking tissue biopsy samples using embodiments of balloon devices in accordance with the invention:

FIG. 7C is a diagram illustrating a method for taking tissue biopsy samples using embodiments of balloon devices in accordance with the invention;

FIG. 8A is a diagram illustrating a method for taking tissue biopsy samples using embodiments of balloon devices in accordance with the invention;

FIG. 8B is a diagram illustrating a method for taking tissue biopsy samples using embodiments of balloon devices in accordance with the invention:

FIG. 8C is a diagram illustrating a method for taking tissue biopsy samples using embodiments of balloon devices in accordance with the invention:

FIG. 9 is a diagram illustrating a method for taking tissue biopsy samples using embodiments of balloon devices in accordance with the invention:

FIG. 10A is a diagram illustrating a method of administering clinical agents using embodiments of balloon devices in accordance with the invention;

FIG. 10B is a diagram illustrating a method of administering clinical agents using embodiments of balloon devices in accordance with the invention;

FIG. 10C is a diagram illustrating a method of administering clinical agents using embodiments of balloon devices in accordance with the invention;

FIG. 11A is a diagram illustrating a method of administering clinical agents using embodiments of balloon devices in accordance with the invention;

FIG. 11B is a diagram illustrating a method of administering clinical agents using embodiments of balloon devices in accordance with the invention;

FIG. 11C is a diagram illustrating a method of administering clinical agents using embodiments of balloon devices in accordance with the invention:

FIG. 12A is a diagram illustrating a method for dilating strictures within tubular organs and other hollow areas of the body using embodiments of balloon devices in accordance with the invention;

FIG. 12B is a diagram illustrating a method for dilating strictures within tubular organs and other hollow areas of the body using embodiments of balloon devices in accordance with the invention:

FIG. 12C is a diagram illustrating a method for dilating strictures within tubular organs and other hollow areas of the body using embodiments of balloon devices in accordance with the invention;

FIG. 12D is a diagram illustrating a method for dilating strictures within tubular organs and other hollow areas of the body using embodiments of balloon devices in accordance with the invention:

FIG. 13A is a diagram illustrating a method for resurfacing mucosa using embodiments of balloon devices in accordance with the invention;

FIG. 13B is a diagram illustrating a method for resurfacing mucosa using embodiments of balloon devices in accordance with the invention; and

FIG. 13C is a diagram illustrating a method for resurfacing mucosa using embodiments of balloon devices in accordance with the invention.

### DETAILED DESCRIPTION

While the following describes embodiments of devices and methods according to the present invention, it is understood that this description is to be considered only as illustrative of the principles of the inventions described herein and is not to be limitative thereof. Numerous other variations, all within the scope of the claims, will readily occur to those of ordinary skill in the art.

The definitions and meanings of terms used herein shall be apparent from the description, the figures, and the context in which the terms are used.

In certain embodiments, the invention can be used in one or more tubular organs or hollow areas of the body, including but not limited to the human mouth, oral cavity, nasal cavity, pharynx, larynx, trachea, bronchi, esophagus, stomach, intestines, colon, rectum gynecological organs, bladder, biliary ducts, pancreatic duct, or urethra, etc., or a combination thereof.

In certain embodiments, the invention can include internal stabilizing wires to help the balloon keep its shape.

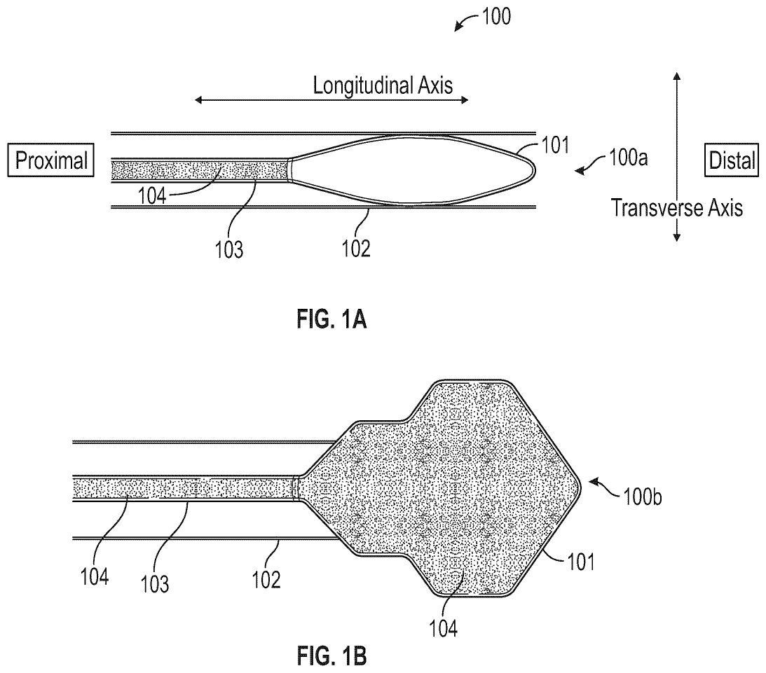

FIGS. 1A and 1B illustrate an embodiment of balloon device 100 that comprises balloon 101, positioning member 102, and hollow member 103. Balloon device 100 may be in deflated state 100a, inflated state 100b, or partially inflated state (not illustrated). Balloon 101, positioning member 102, and hollow member 103 are configured with balloon 101 located distal to positioning member 102 and hollow member 103. Balloon 101 and hollow member 103 are attached such that the hollow member 103 may be used to channel a fluid 104 (via injection, suction, or other appropriate means) to balloon 101 to inflate balloon 101 and from balloon 101 to deflate balloon 101. Hollow member 103 is further operable to extend, position, and retract the balloon. Positioning member 102 is operable to position hollow member 103 and balloon 101 and to control the positional direction of balloon 101, Balloon 101 is inflatable to differing diameters along its length (i.e. along its longitudinal axis) to allow balloon device 100 to access different depths of a target tissue when balloon 101 makes contact with a target tissue.

In the fully inflated state, the length of balloon 101 will vary depending on the intended application of balloon device 100. Further, balloon 101 in the fully inflated state may have a different diameter at various points along its longitudinal axis depending on the intended application of balloon device 100. By way of example, for vascular applications, embodiments of balloon 101 in the fully inflated state may have a maximum diameter in the range of about 2 mm to 30 mm and a length in the range of about 2 mm to 30 cm. In another example, for bronchial applications, balloon 101 in the fully inflated state may have a maximum diameter in the range of 4 mm to 10 mm and a length in the range of about 2 mm to 4 cm In a further example, for gastrointestinal applications, balloon 101 in the fully inflated state may have a maximum diameter in the range of about 6 mm to 30 mm and a length in the range of about 1 cm to 10 cm. In yet another example, for biliary applications balloon 101 in the fully inflated state may have a maximum diameter in the range of about 2 mm to 10 mm and a length in the range of about 2 mm to 5 cm. In another example, for coronary applications, balloon 101 in the fully inflated state may have a maximum diameter in the range of about 2 mm to 6 mm and a length in the range of about 2 nm to 2 cm. And for urinary applications, balloon 101 in the fully inflated state may have a maximum diameter in the range of about 2 mm to 6 mm and a length in the range of about 2 mm to 4 cm. Other applications are contemplated, and the appropriate widths and lengths of balloon 101 may be selected accordingly to achieve an appropriate tissue depth depending on the application.

In certain embodiments, balloon 101 may comprise a material such as medical grade plastic, silicone, latex, polyester, nylon, or polyurethane, etc., or a combination thereof. In certain embodiments, balloon 101 when inflated is in the range of about 2 mm to 300 mm in length with a maximum diameter between 2 mm and 30 mm Positioning member 102 may comprise a catheter, endoscope, lumen, sheath, forceps, or snare, etc., or a combination thereof. In certain embodiments, hollow member 103 may comprise a material such as medical grade plastic, silicone, latex, or polyurethane, etc., or a combination thereof In certain embodiments, fluid 104 may comprise sterile water, saline, liquid nitrogen, contrast, radiopaque fluid, carbon dioxide, or oxygen, etc., or a combination thereof.

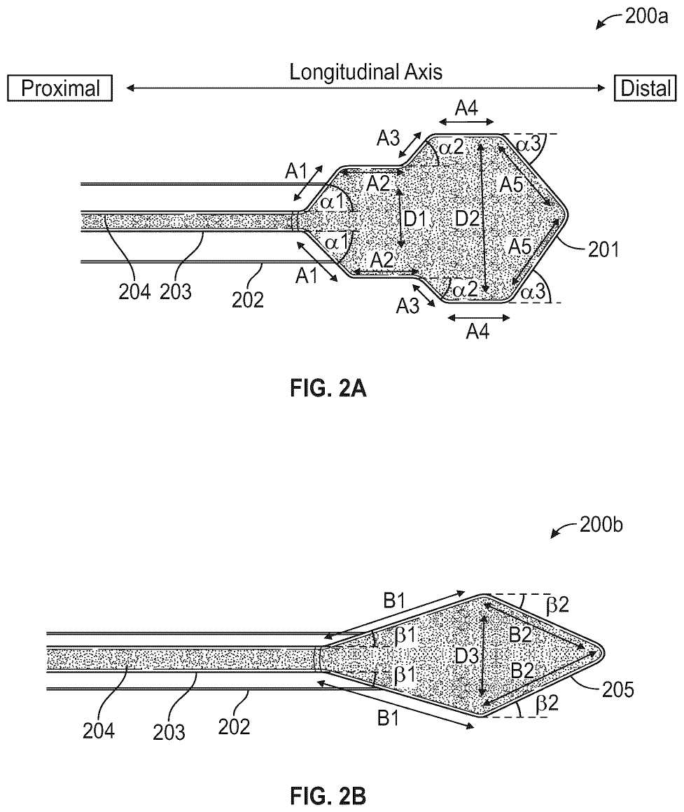

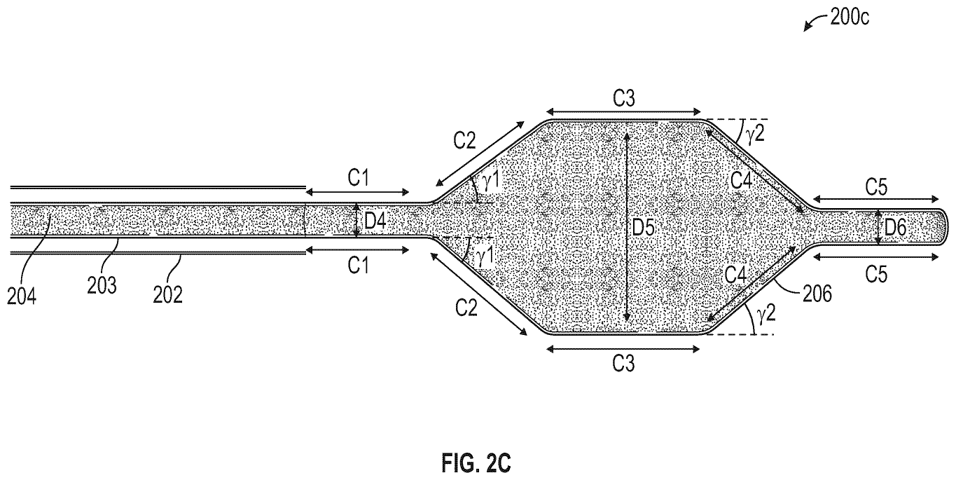

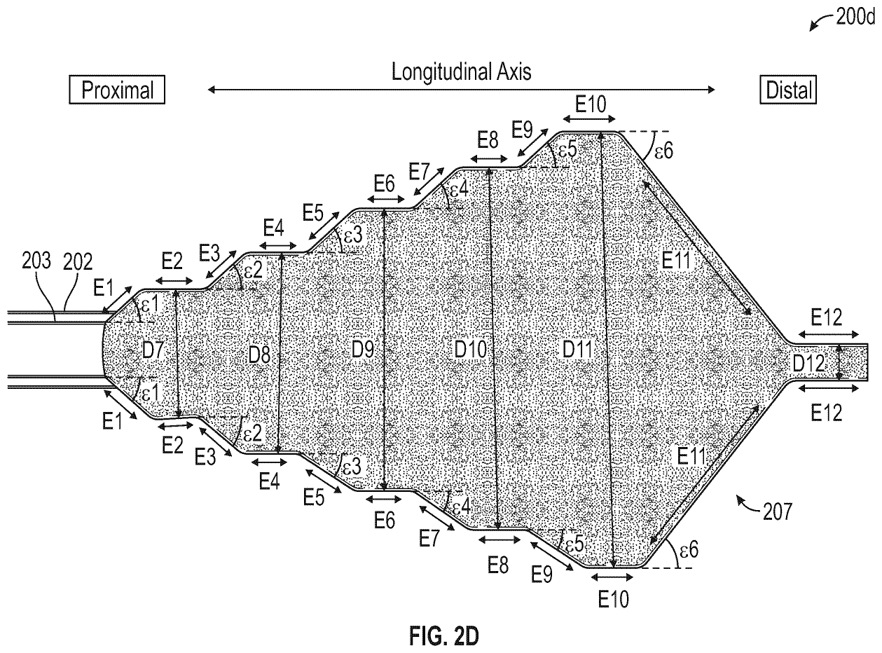

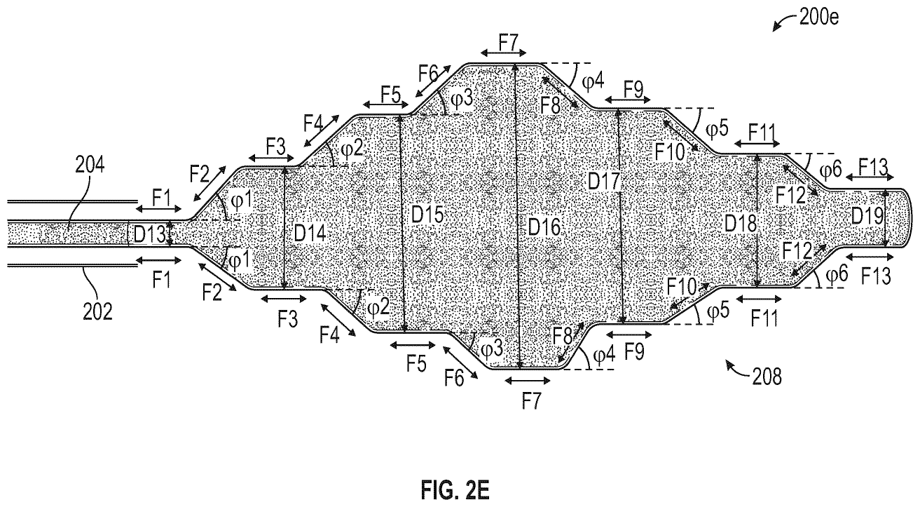

Turning now to FIGS. 2A, 2B, and 2C, embodiments of balloon devices (200a, 200b, 200c) comprising balloon (201, 205, 206), positioning member 202, hollow member 203, and fluid 204 are shown. In balloon device 200a, inflated balloon 201 comprises a proximal first segment A1 that has larger diameter closer to its distal end such that this first segment A1 forms a first angle α1 outwards from the longitudinal axis. Second segment A2 is located distal to first segment A1 and has a diameter D1. Third segment A3 is positioned distal to second segment A2 and has a larger diameter closer to its distal end such that this third segment A3 forms a second angle α2 outwards from the longitudinal axis. Fourth segment A4 is positioned distal to third segment A3 and has a diameter D2. Fifth segment A5 is positioned distal to fourth segment A4 and has a smaller diameter closer to its distal end such that this fifth segment A5 forms a third angle α3 inwards towards the longitudinal axis.

In balloon device 200b, inflated balloon 205 comprises a proximal first segment B1 that has a has a larger diameter closer to its distal end such that this first segment B1 forms a first angle β1 outwards from the longitudinal axis and a distal second segment B2 that has a smaller diameter closer to its distal end such that this second segment B2 forms a second angle β2 inwards towards the longitudinal axis. Balloon device 200b has a maximum diameter D3.

In balloon device 200c, inflated balloon 206 comprises a proximal first segment C1 that has a diameter D4, a second segment C2 that is distal to the first segment C1 and has a has a larger diameter closer to its distal end such that this second segment C2 forms a first angle γ1 outwards from the longitudinal axis, a third segment C3 that is distal to the second segment C2 and has a diameter D5, a fourth segment C4 that is distal to the third segment C3 and has a smaller diameter closer to its distal end such that this fourth segment C4 forms a second angle γ2 inwards towards the longitudinal axis, and a distal fifth segment C5 that is distal to the fourth segment C4 and has a diameter D6.

In certain embodiments of balloon device 200a, the first segment A1 is in the range of about 0.5 to 29.5 mm in length, first angle α1 is in the range of about 30 to 45 degrees, second segment A2 is in the range of about 0.5 to 297.7 mm in length, diameter D1 is in the range of about 0.5 to 29.5 mm, third segment A3 is in the range of about 0.5 to 29.5 mm in length, second angle α2 is in the range of about 30 to 45 degrees, fourth segment A4 is in the range of about 0.5 to 297.7 mm in length, diameter D2 is in the range of about 2 to 30 mm, fifth segment A5 is in the range of about 1.4 to 30 mm in length, and third angle 3 is in the range of about 30 to 45 degrees. Balloon 201 may have a total length in the range of about 3.4 mm to 30 cm. In certain embodiments of balloon device 200b, the first segment B1 is in the range of about 1.4 to 30 mm in length, first angle γ1 is in the range of about 30 to 45 degrees, diameter D3 is in the range of about 2 to 30 mm, second segment B2 is in the range of about 1.4 to 30 mm in length, and second angle 12 is in the range of about 30 to 45 degrees. Balloon 205 may have a total length in the range of about 2.8 to 52 mm. In certain embodiments of balloon device 200c, the first segment C1 is in the range of about 0.5 to 297 mm in length, diameter D4 is in the range of about 0.5 to 29.5 mm, second segment C2 is in the range of about 1 to 29.5 mm in length, first angle γ1 is in the range of about 30 to 45 degrees, third segment C3 is in the range of about 0.5 to 297 mm in length, diameter D5 is in the range of about 2 to 30 mm, fourth segment C4 is in the range of about 1 to 29.5 mm in length, second angle 72 is in the range of about 30 to 45 degrees, fifth segment C5 is in the range of about 0.5 to 297 mm in length, and diameter D6 is in the range of about 0.5 to 29.5 mm. Balloon 206 may have a total length in the range of about 3.6 mm to 30 cm.

In some embodiments, a balloon may have a region of maximum diameter, as illustrated by balloon device 200a, or a point of maximum diameter, as illustrated by balloon device 200b. In some embodiments, a, balloon may vary in diameter in a step-wise fashion along its longitudinal axis, as illustrated by balloon device 200a, or continuously, as illustrated by balloon device 200b, or a combination thereof. In some embodiments, a balloon may have maximum diameter toward its distal end, as illustrated by balloon device 200a, a maximum diameter toward the center of its longitudinal axis, as illustrated by balloon device 200b, or a maximum diameter near its proximal end.

Turing now to FIGS. 2D and 2E, embodiments of balloon devices (200d, 200e) comprising balloon (207, 208), positioning member 202, hollow member 203, and fluid 204 are shown. In balloon device 200d, inflated balloon 207 comprises proximal first segment E1 that has a larger diameter closer to its distal end such that this first segment E1 forms a first angle ε1 outwards from the longitudinal axis. Second segment E2 is positioned distal to the first segment E1 and has a diameter D7. Third segment E3 is positioned distal to second segment E2 and has a larger diameter closer to its distal end such that this third segment E3 forms a second angle ε2 outwards from the longitudinal axis. Fourth segment E4 is positioned distal to third segment E3 and has a diameter D8. Fifth segment E5 is positioned distal to fourth segment E4 and has a larger diameter closer to its distal end such that this fifth segment E5 forms a third angle ε3 outwards from the longitudinal axis. Sixth segment E6 that is positioned distal to fifth segment E5 and has a diameter D9. Seventh segment E7 is positioned distal to sixth segment E6 and has a larger diameter closer to its distal end such that this seventh segment E7 forms a fourth angle ε4 outwards from the longitudinal axis. Eighth segment E8 is positioned distal to the seventh segment E7 and has a diameter D10. Ninth segment E9 is positioned distal to eighth segment E8 and has a larger diameter closer to its distal end such that this ninth segment E9 forms a fifth angle ε5 outwards from the longitudinal axis. Tenth segment E10 is positioned distal to ninth segment E9 and has a diameter D11. Eleventh segment E11 is positioned distal to tenth segment E10 and has a smaller diameter closer to its distal end such that this eleventh segment E11 forms a sixth angle ε6 inwards towards the longitudinal axis, Twelfth segment E12 is positioned distal to segment E11 and has a diameter D12.

In balloon device 200e, inflated balloon 208 comprises a proximal first segment F1 that has a diameter D13. Second segment F2 is positioned distal to first segment F1 and has a larger diameter closer to its distal end such that this second segment F2 forms a first angle φ1 outwards from the longitudinal axis. Third segment F3 is positioned distal to second segment F2 and has a diameter D14. Fourth segment F4 is positioned distal to third segment F3 and has a larger diameter closer to its distal end such that this fourth segment F4 forms a second angle φ2 outwards from the longitudinal axis. Fifth segment F5 is positioned distal to fourth segment F4 and has a diameter D15. Sixth segment F6 is positioned distal to fifth segment F5 and has a larger diameter closer to its distal end such that this sixth segment F6 forms a third angle 93 outwards from the longitudinal axis. Seventh segment F7 is positioned distal to the sixth segment F6 and has a diameter D16. Eighth segment F8 is positioned distal to the seventh segment F7 and has a smaller diameter closer to its distal end such that this eighth segment F8 forms a fourth angle θ4 inwards towards the longitudinal axis. Ninth segment F9 is positioned distal to the eighth segment F8 and has a diameter D17. Tenth segment F10 is distal to ninth segment F9 and has a smaller diameter closer to its distal end such that this tenth segment F10 forms a fifth angle φ5 inwards towards the longitudinal axis. Eleventh segment F11 is distal to tenth segment F10 and has a diameter D18. Twelfth segment F12 is positioned distal to eleventh segment F11 and has a smaller diameter closer to its distal end such that this twelfth segment F12 forms a sixth angle φ6 inwards towards the longitudinal axis. Thirteenth segment F13 is positioned distal to twelfth segment F12 and has a diameter D19.

In certain embodiments of balloon device 200d, first segment E1 is in the range of about 0.5 to 28 mm in length, first angle si is in the range of about 30 to 45 degrees, second segment E2 is in the range of about 0.5 to 296.2 mm in length, diameter D7 is in the range of about 0.5 to 28 mm, third segment E3 is in the range of about 0.5 to 28 mm in length, second angle ε2 is in the range of about 30 to 45 degrees, fourth segment E4 is in the range of about 0.5 to 296.2 mm in length, diameter D8 is 1 to 28.5 mm, fifth segment E5 is in the range of about 0.5 to 28 mm in length, third angle α3 is in the range of about 30 to 45 degrees, sixth segment E6 is in the range of about 0.5 to 296.2 mm in length, diameter D9 is in the range of about 1.5 t 29 mm, seventh segment E7 is in the range of about 0.5 to 28 mm in length, fourth angle ε4 is in the range of about 30 to 45 degrees, eighth segment E8 is in the range of about 0.5 to 296.2 mm in length, diameter D10 is in the range of about 2 to 29.5 mm, ninth segment E9 is in the range of about 0.5 to 28 mm in length, fifth angle α5 is in the range of about 30 to 45 degrees, tenth segment E10 is in the range of about 0.5 to 296.2 mu in length, diameter D11 is about 2.5 to 30 mm, eleventh segment E11 is in the range of about 1.7 to 29.5 mm in length, sixth angle α5 is in the range of about 30 to 45 degrees, twelfth segment E12 is in the range of about 0.5 to 296.2 mm in length, and diameter D12 is about 0.5 to 29.5 mm. Balloon 207 may have a total length in the range of about 4.5 mm to 30 cm.

In certain embodiments of balloon device 200e, the first segment F is in the range of about 0.5 to 294.8 mm in length, diameter D13 is in the range of about 0.5 to 28.5 mm, second segment F2 is in the range of about 0.5 to 28 mm in length, first angle φ1 is in the range of about 30 to 45 degrees, third segment F3 is in the range of about 0.5 to 294.8 mm in length, diameter D14 is in the range of about 1 to 29 mm, fourth segment F4 is in the range of about 0.5 to 28 mm in length, second angle φ² is in the range of about 30 to 45 degrees, fifth segment F5 is in the range of about 0.5 to 294.8 mm in length, diameter D15 is in the range of about 1.5 to 29.5 mm, sixth segment F6 is in the range of about 0.5 to 28 mm in length, third angle φ3 is in the range of about 30 to 45 degrees, seventh segment F7 is in the range of about 0.5 to 294.8 mm in length, diameter D16 is in the range of about 2 to 30 mm, eighth segment F8 is in the range of about 0.5 to 28 mm in length, fourth angle φ4 is in the range of about 30 to 45 degrees, ninth segment F9 is in the range of about 0.5 to 294.8 mm in length, diameter D17 is in the range of about 1.5 to 29.5 mm, tenth segment F10 is in the range of about 0.5 to 28 mm in length, fifth angle φ5 is in the range of about 30 to 45 degrees, eleventh segment F11 is in the range of about 0.5 to 294.8 mm in length, diameter D18 is in the range of about 1 to 29 mm, twelfth segment F12 is in the range of about 0.5 to 28 mm in length, sixth angle φ6 is in the range of about 30 to 45 degrees, thirteenth segment F13 is in the range of about 0.5 to 294.8 run in length, and diameter D19 is in the range of about 0.5 to 28.5 mm. Balloon 208 may have a total length in the range of about 5 mm to 30 cm.

In certain further embodiments of the invention, the balloon may comprise: a proximal first segment that is about 0.5 to 29 mm in length with a distally increasing diameter and wherein the proximal first segment forms an angle of about 30 to 45 degrees outwards from the longitudinal axis of the balloon; a second segment, located distal to the proximal first segment, that is about 0.5 to 297.9 mm in length with a diameter of about 0.5 to 29 mm; a third segment, located distal to the second segment, that is about 0.5 to 29 mm in length with a distally increasing diameter and wherein the third segment forms an angle of about 30 to 45 degrees outwards from the longitudinal axis of the balloon; a fourth segment, located distal to the third segment, that is about 0.5 to 297.9 mm in length with a diameter of about 1 to 29.5 mm; a fifth segment, located distal to the fourth segment, that is about 0.5 to 29 mm in length with a distally increasing diameter and wherein the fifth segment forms an angle of about 30 to 45 degrees outwards from the longitudinal axis of the balloon; a sixth segment, located distal to the fifth segment, that is about 0.5 to 297.9 mm in length with a diameter of about 2 to 30 mm; and a distal seventh segment, located distal to the sixth segment, that is about 1.4 to 30 mm in length with a distally decreasing diameter and wherein the distal seventh segment forms an angle of about 30 to 45 degrees inwards towards the longitudinal axis of the balloon.

In certain further embodiments of the invention, the balloon may comprise: a proximal first segment that is about 0.5 to 296.5 mm in length with a diameter of about 0.5 to 29.0 mm; a second segment, located distal to the proximal first segment, that is about 0.5 to 28.5 mm in length with a distally increasing diameter and wherein the second segment forms an angle of about 30 to 45 degrees outwards from the longitudinal axis of the balloon; a third segment, located distal to the second segment, that is about 0.5 to 296.5 mm in length with a diameter of about 1 to 29.5 mm; a fourth segment, located distal to the third segment, that is about 0.5 to 28.5 mm in length with a distally increasing diameter and wherein the fourth segment forms an angle of about 30 to 45 degrees outwards from the longitudinal axis of the balloon; a fifth segment, located distal to the fourth segment, that is about 0.5 to 296.5 mm in length with a diameter of about 2 to 30 mm; a sixth segment, located distal to the fifth segment, that is about 0.5 to 28.5 mm in length with a distally decreasing diameter and wherein the sixth segment forms an angle of about 30 to 45 degrees inwards towards the longitudinal axis of the balloon; a seventh segment, located distal to the sixth segment, that is about 0.5 to 296.5 mm in length with a diameter of about 1 to 29.5 mm; an eighth segment, located distal to the seventh segment, that is about 0.5 to 28.5 mm in length with a distally decreasing diameter and wherein the eighth segment forms an angle of about 30 to 45 degrees inwards towards the longitudinal axis of the balloon; and a distal ninth segment, located distal to the eighth segment, that is about 0.5 to 296.5 mm in length with a diameter of about 0.5 to 29 mm,

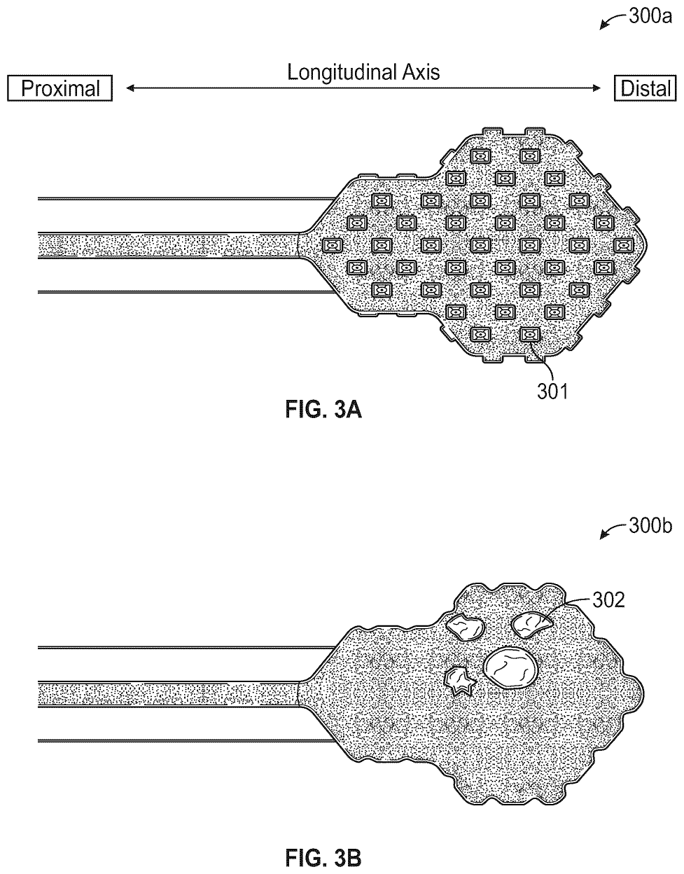

FIGS. 3A and 3B show embodiments of balloon devices (300a, 300b) with balloons of varying diameter and textured surfaces (301, 302) useful for tissue biopsy, stricture dilation, mucosal resurfacing and/or clinical agent administration. Textured surface 301 may comprise raised shapes, as illustrated by balloon device 300a. Textured surface 302 may comprise indentations or depressions in the form of shapes, as in balloon device 300b.

In certain embodiments, textured surface 301, 302 may comprise raised shapes, indentations or depressions in the form of shapes, or combinations thereof. In certain embodiments, the textured surface of a balloon ma comprise geometric shapes, as in balloon device 300a, freeform shapes, as in balloon device 300b, or a combination thereof. In certain embodiments of the invention, the textured surface of a balloon may also comprise shapes of equal size, as in balloon device 300a, or shapes of different sizes, as in balloon device 300b. Textured surfaces 301, 302 may be positioned symmetrically or asymmetrically, and on part (as shown in balloon device 300b) or all (as shown in balloon device 300a) of the surface of the balloon. In some embodiments, the height of the textured surface may correlate with the depth of tissue that will be in contact with the balloon.

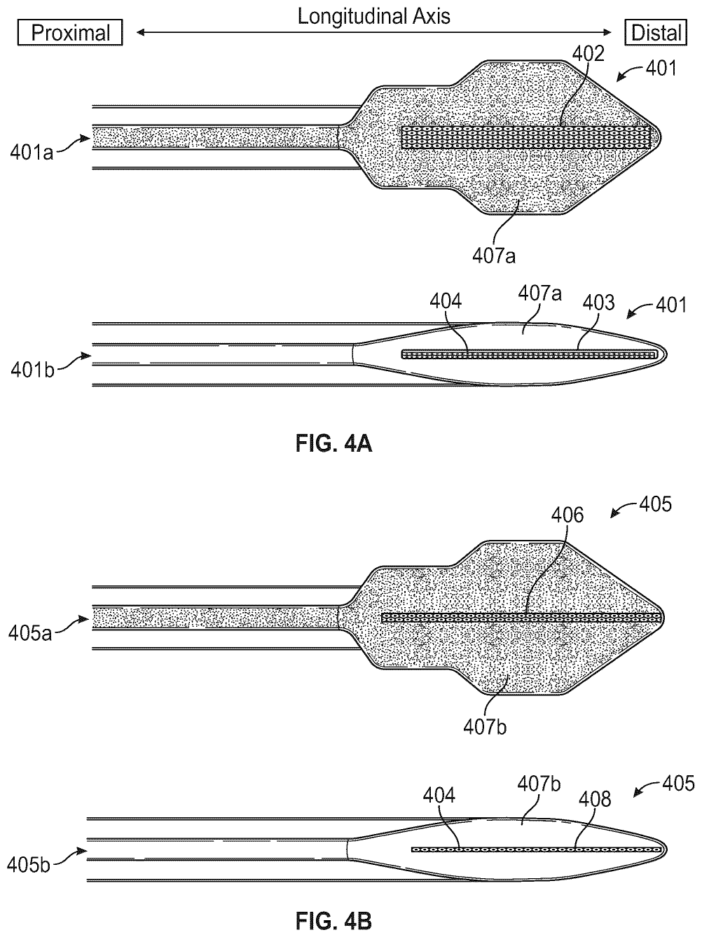

Turning now to FIGS. 4A and 4B, balloon devices (401, 405) comprising balloons of varying diameter and textured surfaces (407a, 407b) useful for tissue biopsy, stricture dilation, mucosal resurfacing and/or clinical agent administration are shown. In balloon device 401, for example, textured surface 407a is illustrated comprising at least one thick lateral streak 402. In certain embodiments, textured surface 407a may comprise multiple thick lateral streaks 402 (not illustrated). Balloon device 401 has an inflated state 401a and deflated state 401b. In the deflated state 401b, at least one thick lateral streak 402 of balloon device 401 forms a depression or well 403 that is operable to receive and hold material 404 for collection from or administration/delivery to a target tissue.

In balloon device 405, textured surface 407b comprises at least one silk thread 406 affixed to balloon device 405, disposed along the longitudinal axis. In certain embodiments, textured surface 407b comprises multiple silk threads 406 (not illustrated). In certain embodiments, at least one silk thread 406 is affixed by means of weaving. Balloon device 405 has an inflated state 405a and a deflated state 405b. In the deflated state 405b, at least one silk thread 406 of balloon device 405 forms a well 408 that is operable to receive and hold material 404 for collection from or administration/delivery to a target tissue.

In some embodiments, the balloon device and/or the textured surface of the balloon comprises one or more materials, such as clinical agents, diagnostic agents, imaging agents, staining agents, visualization agents, detection agents, treatment agents, cytotoxic agents, immunological agents, antimicrobial agents, radioactive agents, tissue-bulking agents, sclerosing agents, acidic agents, timed-release chemical agents, stimulant agents, fibrosis-inducing agents, anti-platelet agents, chemotherapeutic agents, marking agents, etc., or a combination thereof.

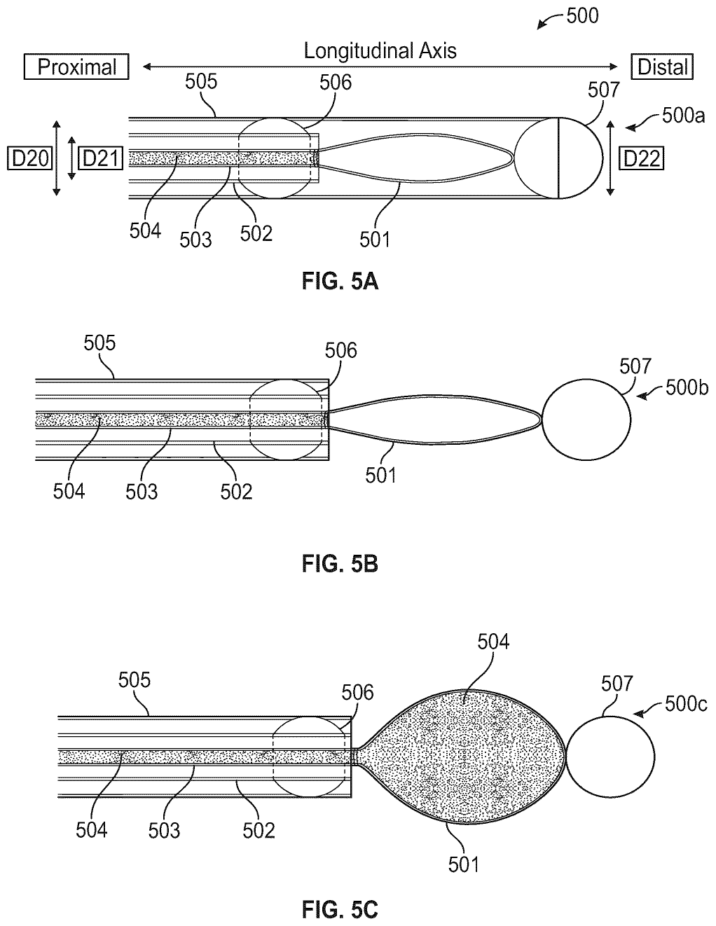

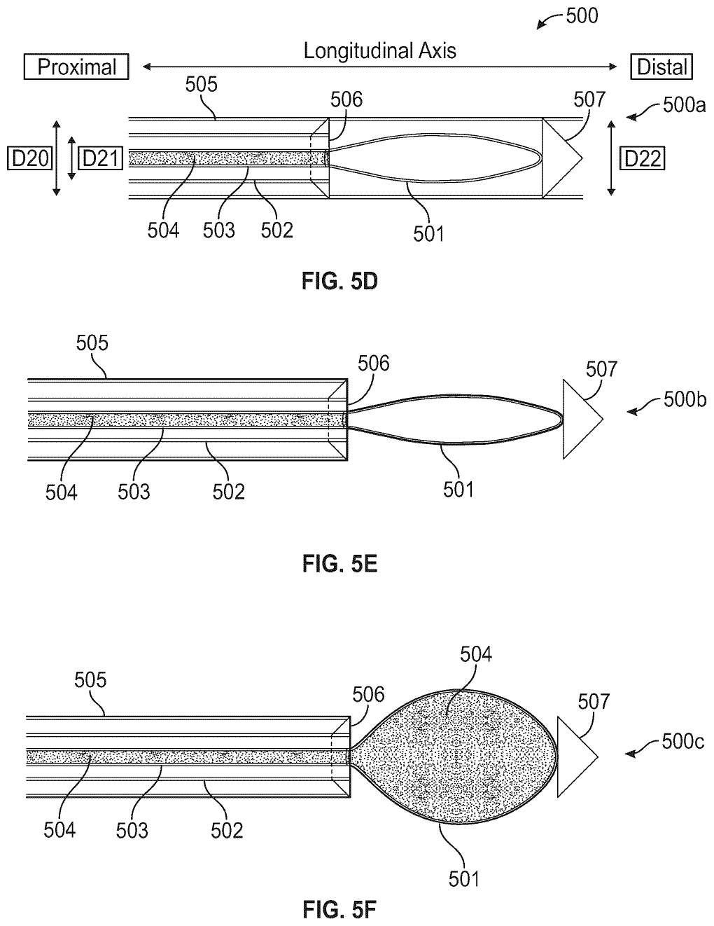

In certain embodiments of the invention, the balloon device may include a sheath and geometrically shaped (e.g., pyramids, spheres, etc.) stoppers that encase the balloon and contain a material (such as a clinical or diagnostic agent or other material described herein) in or on the balloon while the balloon is in the sheath. For example, turning to FIGS. 5A-F, an embodiment of balloon device 500 comprising sheath 505 and geometrically shaped stoppers (506, 507) operable with balloon 501 to contain a material in or on balloon device 500 is shown. Balloon device 500 comprises balloon 501, positioning member 502, hollow member 503, sheath 505, proximal geometrically shaped stopper 506, and distal geometrically shaped stopper 507. Geometrically shaped stoppers 506, 507 may be any shape known to those of ordinary skill in the art and can include spheres, pyramids, cubes, and others. Balloon device 500 can be in deflated retracted state 500a, deflated protruding state 500b, or partially or fully inflated protruding state 500c. Proximal geometrically shaped stopper 506 and distal geometrically shaped stopper 507 are illustrated as spherical in shape in FIGS. 5A-C and pyramids in FIGS. 5D-F. In this particular embodiment, balloon 501, positioning member 502, hollow member 503, proximal geometrically shaped stopper 506, and distal geometrically shaped stopper 507 are attached (here, along a longitudinal axis) with balloon 501 being positioned proximal to the distal geometrically shaped stopper 507 and being positioned distal to the positioning member 502, hollow member 503, and proximal geometrically shaped stopper 506. Balloon 501, positioning member 502, hollow member 503, proximal geometrically shaped stopper 506, and distal geometrically shaped stopper 507 are attached such that the hollow member 503 is operable to permit a fluid 504 to enter and inflate balloon 501 and be removed from and deflate balloon 501, Positioning member 502 is operable to manipulate the position of balloon 501. Sheath 505 has an outer diameter D20 and inner diameter D21, Proximal geometrically shaped stopper 506 and distal geometrically shaped stopper 507 each have a diameter D22.

In certain embodiments of the invention, sheath 505 comprises a material such as medical grade silicone, latex, or plastics, etc., or a combination thereof. Geometrically shaped stoppers 506, 507 may comprise a material such as medical grade silicone, latex, or plastics, etc., or a combination thereof.

In an exemplary embodiment, outer diameter of sheath D20 is in the range of about 0.5 mm to 10 mm, inner diameter of sheath D21 is in the range of about 0.25 mm to 9.75 mm, and geometrically shaped stopper diameter D22 is in the range of about 0.25 mm to 9.75 mm. In an additional exemplary embodiment, outer diameter of sheath D20 is in the range of about 0.5 mm to 5 mm and inner diameter of sheath D21 is in the range of about 0.25 mm to 4 mm. In further embodiments, the maximum diameter of the geometrically shaped stoppers is less than the inner diameter of the sheath so that the geometrically shaped stoppers can be extended out from the sheath and then retracted back into the sheath. In even further embodiments, the geometrically shaped stoppers comprise flexible material that compresses when retracted within the balloon device and expands on its own or with the balloon upon inflation.

In alternate embodiments, a balloon, positioning member, hollow member, proximal geometrically shaped stopper, and distal geometrically shaped stopper are arranged such that a sheath, the proximal geometrically shaped stopper, and the distal geometrically shaped stopper encase the balloon when the balloon is in a deflated state and retracted within the balloon device. In this embodiment, the balloon device is operable to contain material in or on the deflated balloon and allow for extension of the balloon distally and inflation. If such a balloon device is used to collect tissue during a biopsy, this encasement of the balloon in its deflated state by the sheath and geometrically shaped stoppers ensures that collected tissue sample in or on balloon (which is deflated and retracted into the balloon device after tissue sample collection) is not lost while balloon device is removed from the body. This encasement further safeguards the collected tissue sample and prevents contamination by the entry of biological material from neighboring tissue while the balloon device is removed from the body. Additionally, in embodiments where the balloon device is used to administer a clinical agent by the balloon, this encasement minimizes the risk that the clinical agent leaks and/or is lost while balloon device is positioned at a target site, allowing for an effective administration of an amount of clinical agent. This also ensures that the clinical agent is not applied inadvertently to neighboring tissue as the balloon device is positioned.

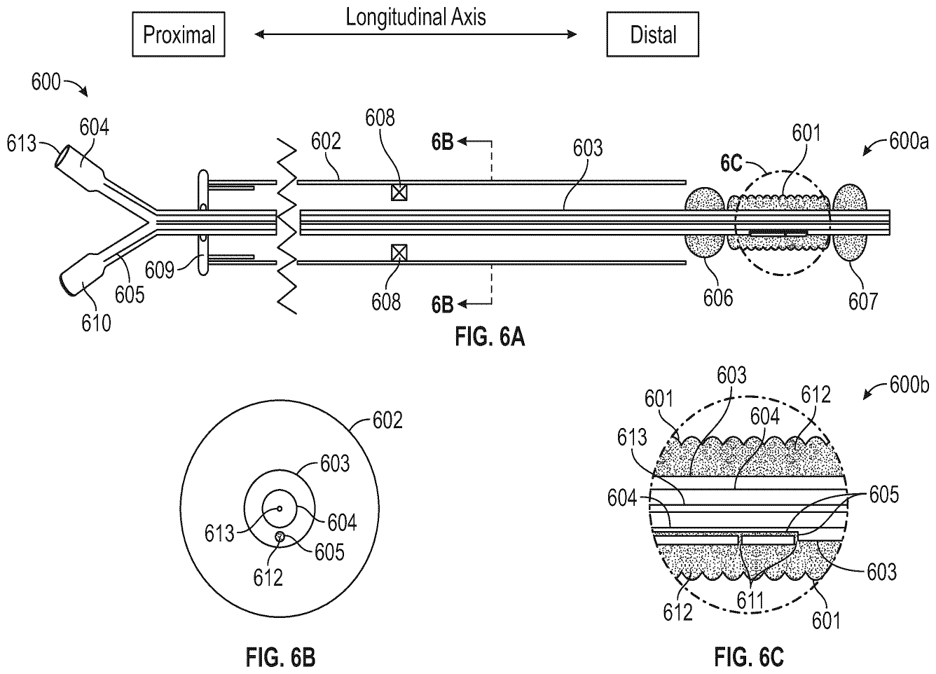

In other embodiments of the invention illustrated in FIGS. 6A-C, balloon device 600 comprises balloon 601 positioning member 602, hollow member 603, guidewire channel or lumen 604, balloon inflation channel 605, and geometrically shaped stoppers (606, 607). Hollow member 603 is operable to extend, position, and retract balloon 601 and to inject and remove a fluid to inflate or deflate balloon 601. Positioning member 602 is operable to control the position and/or direction of balloon 601 and hollow member 603. Positioning member 602 further comprises one or more backstops 608, and plug 609, which provides a proximal seal to prevent loss of fluid or material. Positioning member 602 may further serve as a sheath for balloon 601. Balloon 601, hollow member 603, guidewire channel 604, balloon inflation channel 605, and geometrically shaped stoppers 606, 607 are configured such that positioning member 602, proximal geometrically shaped stopper 606, and distal geometrically shaped stopper 607 encase or otherwise surround balloon 601 when balloon device 600 is in a deflated retracted state.

Balloon device 600 can be in deflated retracted state (not illustrated), or unsheathed and deflated in a protruding state (as illustrated in FIG. 6A), or partially or fully inflated in a protruding state (not illustrated). Hollow member 603 may be a hollow tube, and is operable to unsheathe balloon 601, in other words, to protrude and retract balloon 601 distally relative to positioning member 602.

Balloon inflation channel 605 further comprises fluid port 610 at its proximal end for insertion of fluid into balloon inflation channel 605, and one or more inflation ports 611 disposed at its distal end for introducing the fluid into balloon 601. Balloon 601, hollow member 603, guidewire channel 604, balloon inflation channel 605, and geometrically shaped stoppers 606, 607 are configured such that fluid port 610, balloon inflation channel 605, and inflation ports 611 can be used to selectively inflate or deflate balloon 601 via channel fluid 612 (via injection, suction, or other appropriate means).

Guidewire channel or lumen 604 through which one or more guidewires (e.g., guidewire 613) can extend can further be operable control the position and/or direction of balloon 601. In balloon device 600, guidewire channel 604 and balloon inflation channel 605 are disposed within hollow member 603, which may be a tube having a wall. In other embodiments, guidewire channel 604 and inflation channel 605 may be disposed partially external to hollow member 603. In one embodiment illustrated in FIG. 6A, the proximal end of hollow member 603 divides into two tracks in the shape of a Y with one track containing guidewire channel 604 and the other track containing balloon inflation channel 605. The two tracks converge at a convergence point and thereafter hollow member extends distally as one hollow channel containing both guidewire channel 604 and balloon inflation channel 605.

Cross-section “6B” of balloon device 600 in FIG. 6A is illustrated in FIG. 6B, which shows the wall of positioning member 602, illustrated as a hollow tube or sheath, within which is disposed hollow member 603, also illustrated as a hollow tube or sheath. Within hollow member 603 is disposed guidewire channel 604 and guidewire 613 shown within. Also, balloon inflation channel 605, with fluid 612, is disposed within hollow member 603.

A detail view “6C” of balloon device 600 of FIG. 6A, is illustrated in FIG. 6C. FIG. 6C illustrates a portion of balloon device 600 along the same longitudinal axis illustrated in FIG. 6A Beginning at the top, balloon 601 is shown on the outside of hollow member 603. Fluid 612, which has been carried or transported via balloon inflation channel 605, is introduced to balloon 601 via balloon inflation port 611, which is disposed at the distal end of balloon inflation channel 605, and inflates balloon 601. Balloon inflation port 611 is disposed at the interface among balloon inflation channel 605, balloon 601, and hollow member 603. Within hollow member 603 is shown guidewire channel 604, within which is disposed guidewire 613.

Balloon 601 can comprise any of the balloon features described above and/or may be itself any of the balloon embodiments disclosed herein (e.g., 101, 201, 205, 206, 207, 208, 401, 405, 501, 701, 801, 1001, 1101, 1201, 1301). Balloon 601 can further comprise a textured surface, which can comprise any of the textured surface features described in the embodiments disclosed above, and may be itself any of the textured surface embodiments disclosed herein (e.g., 301, 302, 407a, 407b, 705, 805, 1005, 1105, 1205, 1305). Geometrically shaped stoppers 606, 607 can comprise any of the geometrically shaped stopper features described herein and may be themselves any of the geometrically shaped stopper embodiments disclosed herein (e.g., 506, 507).

In certain embodiments, balloon 601, positioning member 602, hollow member 603, guidewire channel 604, balloon inflation channel 605, geometrically shaped stoppers 606, 607, one or more backstops 608, plug 609, fluid port 610, inflation ports 611, and guidewire 613 may each comprise silicone, latex, polyurethane, plastics, metals, clinical agent, or material that is rigid or flexible, thermally or electromagnetically conducting or insulating or reflective, textured or non-textured, coated (wherein such coating may be clinical agent) or non-coated, ablating or non-ablating, radioactive or non-radioactive, radiopaque or radiolucent, etc., or a combination thereof. In certain embodiments, fluid 612 may comprise a liquid or gas, such as a clinical agent, sterile water, saline, liquid nitrogen, oxygen, etc., or a combination thereof.

In the fully inflated state, the length of balloon 601 will vary depending on the intended use of balloon device 600, Further, balloon 601 in the fully inflated state may have a different diameter at various points along its longitudinal axis depending on the intended use of balloon device 600. By way of example, for vascular applications, embodiments of balloon 601 in the fully inflated state may have a maximum diameter in the range of about 2 mm to 30 mm, and a length in the range of about 2 mm to 30 cm. In another example, for bronchial applications, balloon 601 in the fully inflated state may have a maximum diameter in the range of 4 mm to 10 mm, and a length in the range of about 2 mm to 4 cm. In a further example, for gastrointestinal applications, balloon 601 in the fully inflated state may have a maximum diameter in the range of about 6 mm to 30 mm and a length in the range of about 1 cm to 10 cm. In yet another example, for biliary applications balloon 601 in the fully inflated state may have a maximum diameter in the range of about 2 mm to 10 mm, and a length in the range of about 2 mm to 5 cm. In another example, for coronary applications, balloon 601 in the fully inflated state may have a maximum diameter in the range of about 2 mm to 6 mm and a length in the range of about 2 mm to 2 cm, And for urinary applications, balloon 601 in the fully inflated state may have a maximum diameter in the range of about 2 mm to 6 mm and a length in the range of about 2 mm to 4 cm. Other applications are contemplated, and the appropriate widths and lengths of balloon 601 may be selected accordingly to achieve an appropriate tissue depth sought to be reached with the balloon.

In some embodiments, positioning member 602 is a hollow tube that has a circumference greater than that of hollow member 603. Hollow member 603 may have a length along its longitudinal axis (as shown in FIG. 6A) greater than that of positioning member 602, such that it is able to distally protrude out from and extend beyond the distal end of positioning member 602, which is open or openable, and then be retracted back within or inside the distal end of positioning member 602 in the proximal direction. In some embodiments, hollow member 603 (with balloon 601 and geometrically shaped stoppers 606, 607) may continue to be retracted until one or more backstops 608 and/or plug 609 prevent geometrically shaped stopper 606 from further movement in a proximal direction within positioning member 602.

In even further embodiments, the lengths of positioning member 602 and hollow member 603 will vary depending on the intended application of balloon device 600. For example, positioning member 602 and hollow member 603 may have lengths in the range of about 10 cm to 100 cm. In additional embodiments, the diameters of positioning member 602 and hollow member 603 will vary depending on the intended application of balloon device 600. By way of example, for vascular applications, embodiments of positioning member 602 and hollow member 603 may have diameters in the range of about 2 mm to 30 mm. In another example, for bronchial applications, positioning member 602 and hollow member 603 may have diameters in the range of 4 mm to 10 mm. In a further example, for gastrointestinal applications, positioning member 602 and hollow member 603 may have diameters in the range of about 6 mm to 30 mm. In yet another example, for biliary applications positioning member 602 and hollow member 603 may have diameters in the range of about 2 mm to 10 mm. In another example, for coronary applications, positioning member 602 and hollow member 603 may have diameters in the range of about 2 mm to 6 mm. And for urinary applications, positioning member 602 and hollow member 603 may have diameters in the range of about 2 mm to 6 mm. Other applications are contemplated, and the appropriate widths and lengths of positioning member 602 and hollow member 603 may be selected accordingly to reach a particular location in the body depending on the application.

In an exemplary embodiment, the outer diameter of positioning member 602 is in the range of about 0.5 mm to 10 mm, the inner diameter of hollow member 603 is in the range of about 0.25 mm to 9.75 mm, and the diameter of geometrically shaped stoppers 606, 607 is in the range of about 0.25 mm to 9.75 mm. In further embodiments, the maximum diameter of the geometrically shaped stoppers is less than the inner diameter of the positioning member so that the geometrically shaped stoppers can be extended out from the positioning member and then retracted back into the positioning member. In even further embodiments, the geometrically shaped stoppers comprise flexible material that compresses when retracted within the balloon device and expands on its own or with the balloon upon inflation.

In certain embodiments, the length of guidewire channel 604 is in the range of about 10 cm to 100 cm and the diameter of guidewire channel 604 is in the range of about 0.04 cm to 0.1 cm. In further embodiments, the length of guidewire 613 is in the range of about 80 cm to 450 cm and the diameter of guidewire 613 is in the range of about 0.03 in to 0.09 in. The appropriate widths and lengths of guidewire channel 604 and guidewire 613 may be selected to achieve an appropriate position and direction of balloon device 600 depending on the intended application of balloon device 600. Contemplated applications include vascular, bronchial, gastrointestinal, biliary, coronary, urinary, and other applications.

In certain embodiments, the length of balloon inflation channel 605 is in the range of about 10 cm to 100 cm and fluid port 610, balloon inflation channel 605, and inflation ports 611 allow for inflation and deflation of balloon 101 at a rate in the range of about 0 cc/s to 50 cc/s. The appropriate length of balloon inflation channel 605 and inflation rate of fluid port 610, balloon inflation channel 605, and inflation ports 611 may be selected depending on the intended application of balloon device 600. Contemplated applications include vascular, bronchial, gastrointestinal biliary, coronary, urinary, and other applications.

Also disclosed herein are inventions relating to methods for selective collection of tissue biopsy from different depths of a target tissue. For example, a method for selective collection of tissue biopsy from different depths of a target tissue comprises the steps of positioning a balloon device (e.g., 100) at a target tissue, inflating a balloon, making contact with a target tissue, rotating, extending, and/or retracting balloon one or more times while inflated, deflating the balloon, and harvesting the collected tissue in or on the balloon.

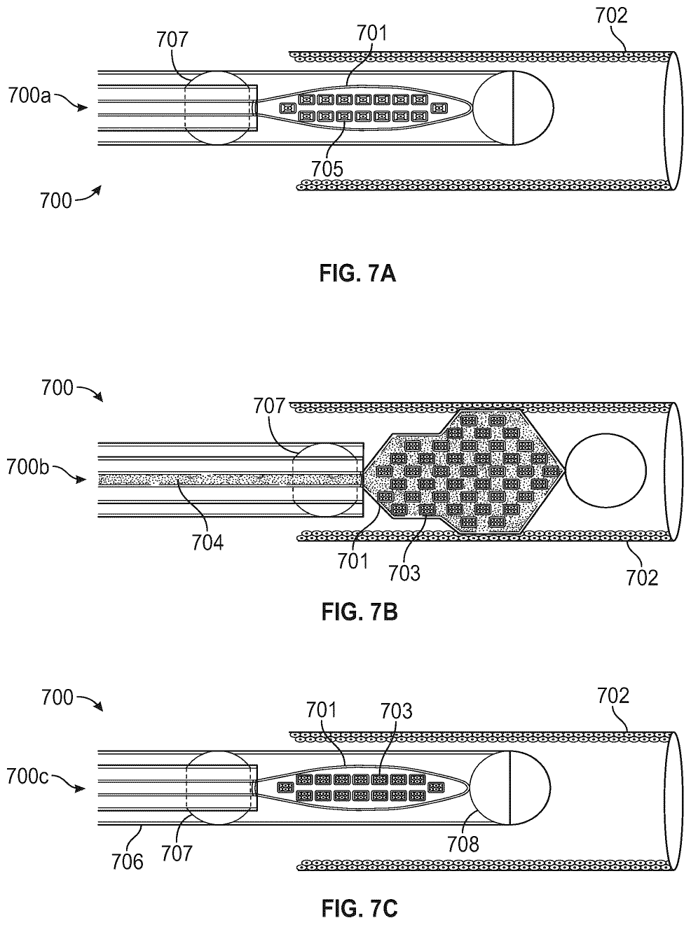

FIGS. 7A-C, for example, illustrate a method of taking a tissue biopsy using an embodiment, balloon device 700. In balloon device 700, balloon 701 is shown having a textured surface 705 with raised shapes (in this exemplary embodiment, abrasive nubs). States 700a and 700b, respectively, illustrate the progression of balloon device 700 relative to tubular organ 702, as balloon device 700 is first positioned at a target tissue, and then balloon 701 is inflated with fluid 704. In inflated state 700b, biopsy 703 tissue sample is collected on the surface of balloon 701. States 700b and 700c, respectively, illustrate the progression of balloon device 700 relative to tubular organ 702 as balloon 701 is then deflated by withdrawing fluid 704 and subsequently retracted into balloon device 700. Finally, balloon device 700 is removed from the target tissue in order to harvest the biopsy 703. In state 700c, biopsy 703 tissue sample is located on balloon 701 and contained by a sheath 706, proximal geometrically shaped stopper 707, and distal geometrically shaped stopper 708. Once balloon device 700 is removed from the body, balloon 701 can be removed from balloon device 700, and biopsy 703 tissue sample can be collected from balloon 701 and analyzed.

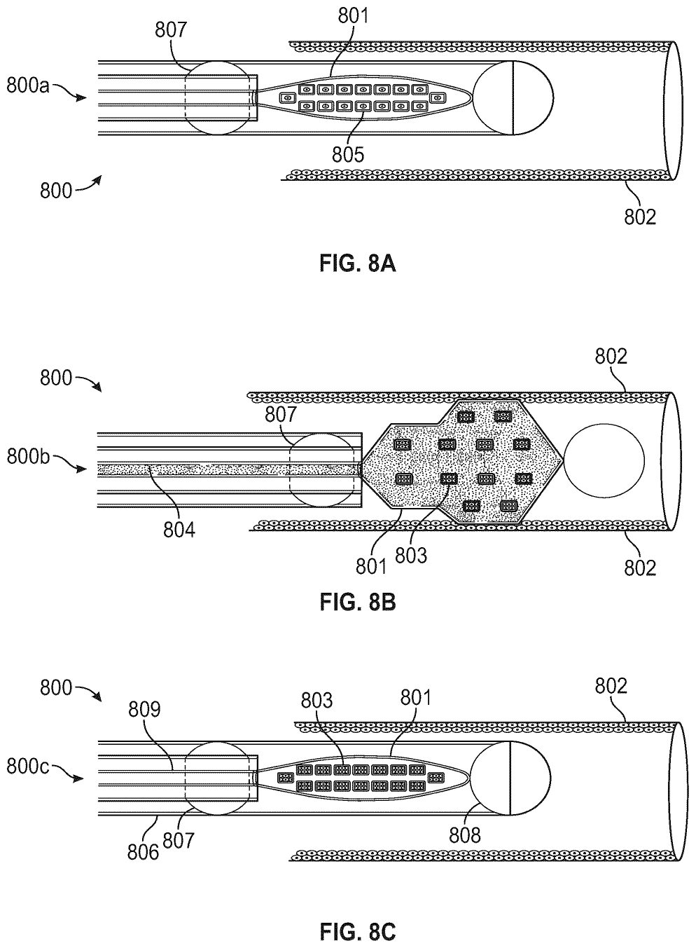

Turning now to FIGS. 8A-C, the method for taking a tissue biopsy discussed above using another embodiment of balloon device 800 is shown. In balloon device 800, balloon 801 comprises a textured surface 805 with indentations and/or depressions in the shape of squares. States 800a and 800b, respectively, illustrate the progression of balloon device 800 relative to tubular organ 802 as balloon device 800 is positioned at a target tissue and then balloon 801 is inflated with fluid 804. In state 800b, biopsy 803 tissue sample has been collected on balloon 801, and in particular, within the indentations or depressions on the surface of balloon 801, after the surface of tubular organ 802 has been abraded by balloon 801. States 800b and 800c, respectively, illustrate the progression of balloon device 800 relative to tubular organ 802 as balloon 801 is deflated and then balloon device 800 is removed from the target tissue in order to harvest the biopsy 803. In state 800c, the biopsy 803 is on balloon 801 and contained by the sheath 806, proximal geometrically shaped stopper 807, and distal geometrically shaped stopper 808.

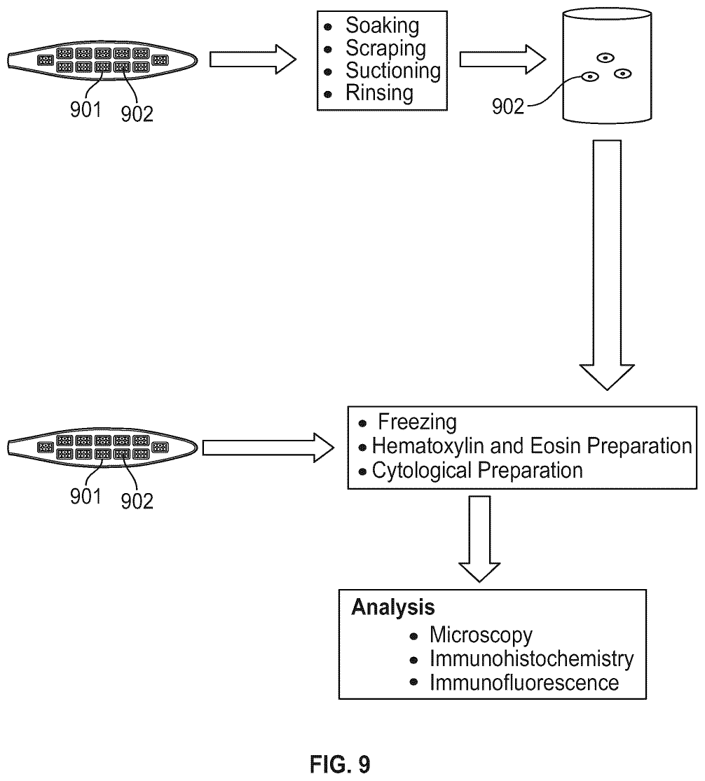

FIG. 9 illustrates an alternate method of taking a tissue biopsy where biopsy 902 tissue sample is collected on balloon 901 and can be isolated via soaking the material and balloon in saline, water, or formaldehyde, etc. or a combination thereof; scraping the material from balloon; suctioning the material from balloon; rinsing the material from balloon with saline, water, or formaldehyde, etc., or a combination thereof; or any combination thereof. Once isolated, the biopsy 902 tissue sample can undergo freezing, hematoxylin and eosin preparation, or cytological preparation, etc., or a combination thereof for analysis via microscopy, immunohistochemistry, or immunofluorescence, etc., or a combination thereof. In certain embodiments, balloon 901 and biopsy 902 tissue sample together can undergo freezing, haematoxylin and eosin preparation, or cytological preparation, etc., or a combination thereof, for analysis via microscopy, immunohistochemistry, or immunofluorescence, etc., or a combination thereof.

In an exemplary embodiment, a method for collecting tissue samples from different depths at a target location comprises the steps of: positioning a balloon device (e.g., 600), at a target location, inflating the balloon with a fluid, contacting a tissue with the balloon, deflating the balloon, retracting the balloon within the positioning member, and harvesting collected tissue from the balloon. An additional method may further comprise the step of dragging and/or rotating the inflated balloon along the target tissue while the balloon is in contact with the tissue.

Also disclosed herein are inventions relating to methods for selective administration of one or more clinical or diagnostic agents to different depths of a target tissue. For example, a method for selective administration of one or more clinical or diagnostic agents to different depths of a target tissue comprises the steps of coating a balloon of a balloon device with one or more clinical agents, positioning the loaded balloon device at a target tissue, extending the balloon coated with the clinical agent, inflating the balloon with a fluid, contacting a target tissue with the balloon to administer the clinical agent to the target tissue, rotating the balloon while in contact with the target tissue, deflating the balloon, and retracting the balloon into the balloon device, wherein the clinical agent is administered to different depths of the tissue.

The one or more clinical agents may comprise a diagnostic agent, imaging agent, staining agent, visualization agent, detection agent, treatment agent, cytotoxic agents, immunological agent, antimicrobial agent, radioactive agent, tissue-bulking agent, sclerosing agent, acidic agent, timed-release chemical agent, stimulant agent, fibrosis-inducing agent, anti-platelet agent, chemotherapeutic agent, or marking agent, etc., or a combination thereof. In certain embodiments, the loading of the balloon device with one or more clinical agents is accomplished by coating the balloon. In another embodiment, the target tissue may be dilated before being contacted by the balloon (for example, if there is a benign or malignant stricture present).

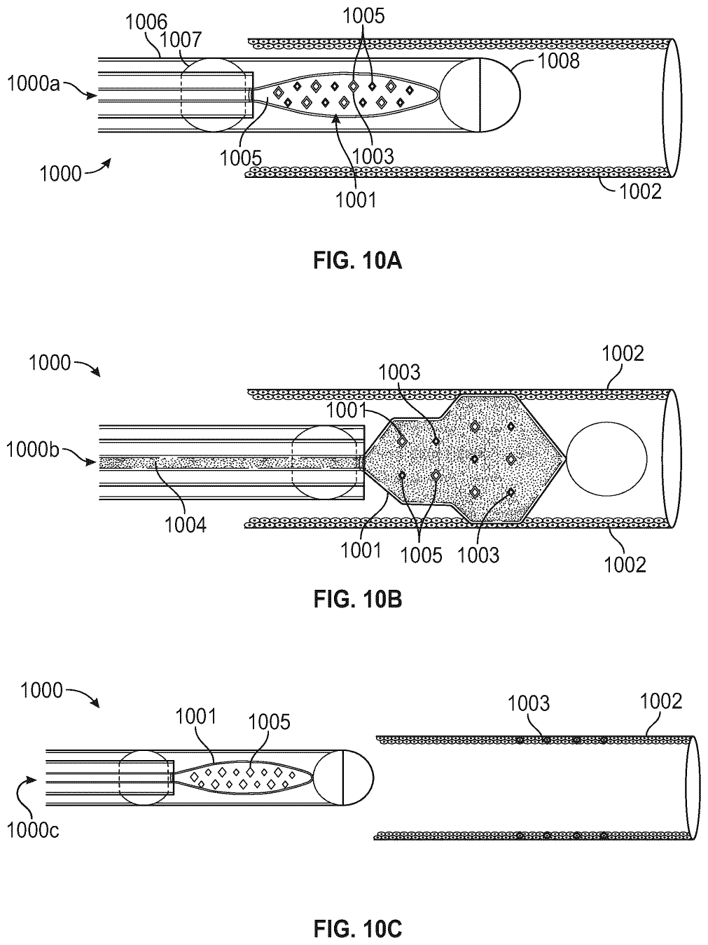

Turning now to FIGS. 10A-C, an embodiment of a method for clinical agent administration is illustrated. In balloon device 1000, balloon 1001 is shown having a textured surface 1005 (in the shape of varying, raised nubs) and is coated with clinical agent 1003. In states 1000a and 1000c, material in or on balloon 1001, including clinical agent 1003, is encased by sheath 1006, proximal geometrically shaped stopper 1007, and distal geometrically shaped stopper 1008. States 1000a and 1000b, respectively, illustrate the progression of balloon device 1000 relative to tubular organ 1002 as balloon device 1000 is positioned at a target tissue and then balloon 1001 extends out from balloon device 1000 and is inflated with fluid 1004. In state 1000b, clinical agent 1003 is administered at the target tissue when the surface of balloon 1001 contacts the target tissue. Because balloon 1001 in this embodiment features varying diameters and a textured surface 1005 having varying heights, balloon 1001 may be applied to target tissue with pressure so that the textured surface reaches varying depths of the target tissue depending on the relative diameters and heights of the textured surface of balloon 1001. In this way, the clinical agent may be selectively administered depending on the configuration of the textured surface of balloon 1001. States 1000b and 1000c, respectively, illustrate the progression of balloon device 1000 after administration relative to tubular organ 1002 (or hollow area of the body) as balloon 1001 is deflated and then removed from the target tissue. In state 1000c, clinical agent 1003 has been administered to the target tissue in a depth-selective manner.

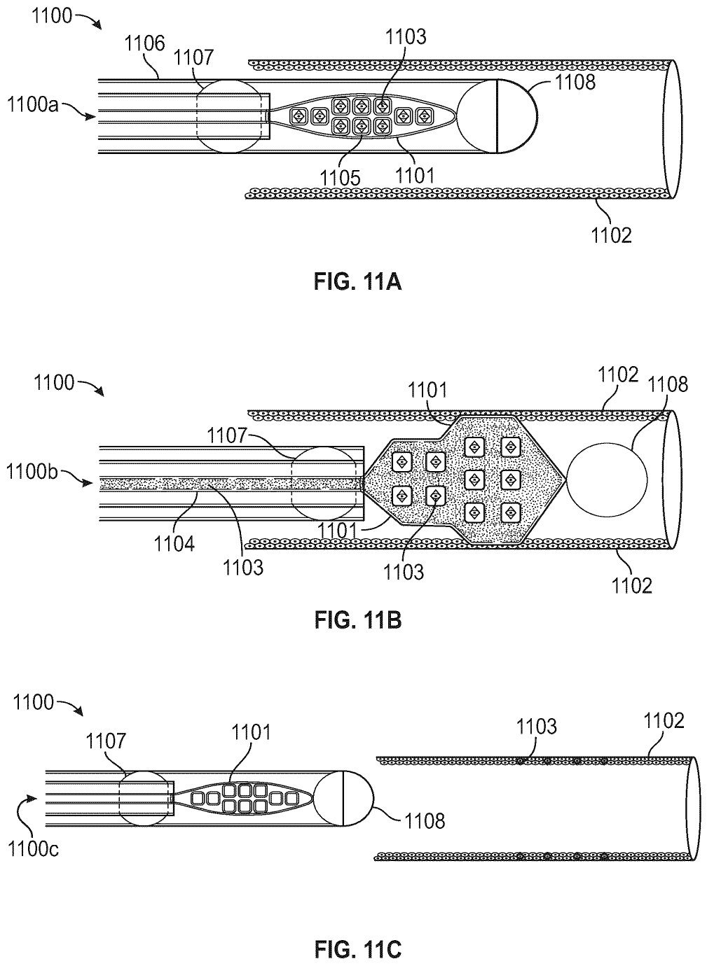

FIGS. 11A-C illustrate a further alternate embodiment of a method for clinical agent administration using balloon device 1100. Here, balloon device 1100 comprises balloon 1101, which has a textured surface 1105 featuring indentations or depressions in the shape of squares. In state 1100a, clinical agent 1103 is coated on the surface of balloon 1101, and is deposited in the indentations or depressions, which act as wells, and encased by sheath 1106, proximal geometrically shaped stopper 1107, and distal geometrically shaped stopper 1108. States 1100a and 1100b, respectively, illustrate the progression of balloon device 1100 relative to tubular organ 1102 as balloon device 1100 is positioned at a target tissue and then balloon 1101 is inflated with fluid through the hollow member 1104. In state 1100b, clinical agent is administered at the target tissue when the surface of balloon 1101 makes contact with the target tissue. States 1100b and 1100c, respectively, illustrate the progression of balloon device 1100 relative to tubular organ 1102 as balloon 1101 is deflated and then balloon device 1100 is removed from the target tissue. In this embodiment, the textured surface of balloon 1101 featuring indentations that act as wells to allow for the administration of larger volumes of clinical agents at a target tissue than would be available on the surface of a smooth balloon.

In an exemplary embodiment, a method of clinical or diagnostic agent administration comprises the steps of positioning a balloon device (e.g., 600) at a target tissue, inflating the balloon with a fluid, contacting a tissue with the balloon, applying the clinical or diagnostic agent to the target tissue, deflating the balloon, and retracting the balloon within the positioning member. An additional method may further comprise the step of dragging and/or rotating the inflated balloon along the target tissue while the balloon is in contact with the tissue.

Also disclosed herein are inventions relating to methods for dilating strictures within tubular organs. For example, a method for dilating strictures within tubular organs comprises the steps of positioning a balloon device (e.g., 100) within a region of tubular organ (or hollow area of the body) affected by a stricture, inflating a balloon, making contact with the stricture and dilating the stricture, deflating the balloon, and retracting the balloon device from the tubular organ. In certain embodiments, the method for dilating strictures within tubular organs can further comprise rotating, extending, or retracting the balloon one or more times while inflated.

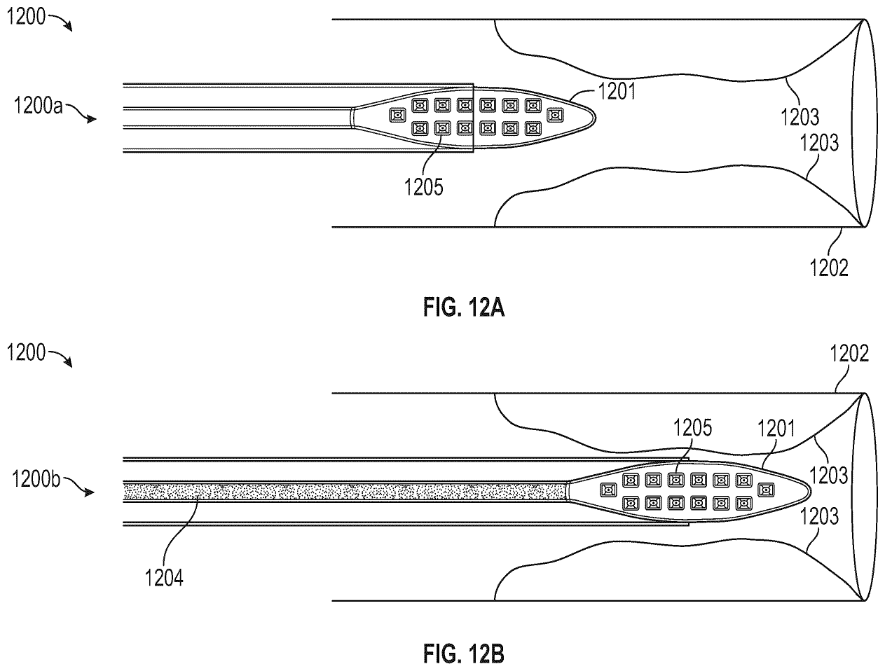

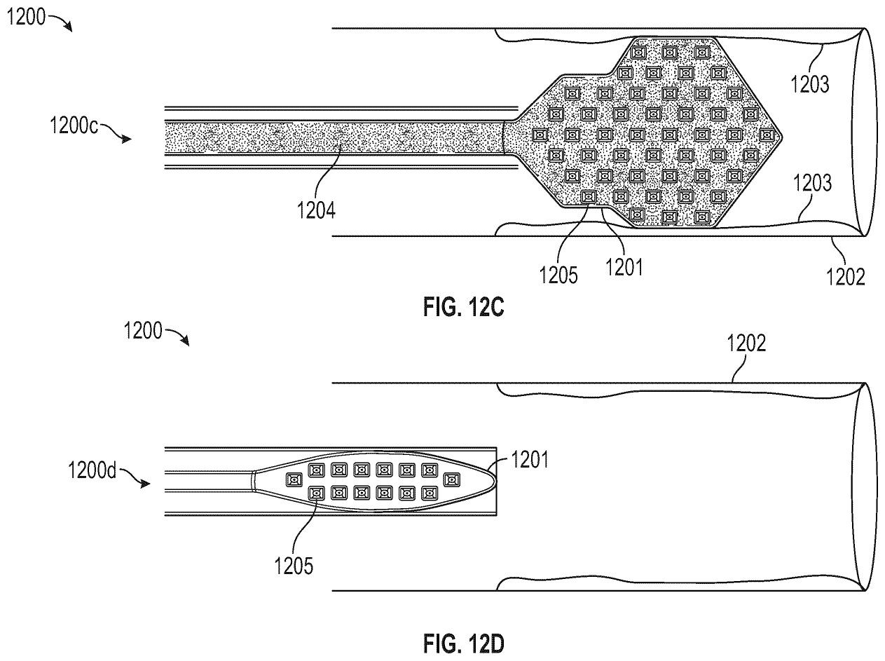

FIGS. 12A-D, for example, illustrate a method of dilating a region of tubular organ or other hollow area of the body affected by a stricture using an embodiment, balloon device 1200. In balloon device 1200, balloon 1201 is shown having a textured surface 1205 with raised shapes. States 1200a and 1200b, respectively, illustrate the progression of balloon device 1200 relative to tubular organ 1202 and stricture 1203, as balloon device 1200 is first positioned inside tubular organ 1202 and then further positioned inside stricture 1203. State 1200c illustrates when balloon 1201 is inflated with fluid 1204. In inflated state 1200c, surface 1205 of balloon 1201 dilates stricture 1203. States 1200c and 1200d, respectively, illustrate the progression of balloon device 1200 relative to tubular organ 1202 and stricture 1203 as balloon 1201 is then deflated by withdrawing fluid 1204 and subsequently retracted into balloon device 1200. Finally, balloon device 1200 is removed from the tubular organ 1202.

In an exemplary embodiment, a method for dilation of a stricture in a tubular organ or other hollow area of the human body comprises the steps of: positioning a balloon device (e.g., 600) on either side of a stricture, extending the hollow member from the distal end of the positioning member through the stricture, inflating the balloon with a fluid, contacting the stricture with the balloon by retracting the hollow member, dilating the stricture, deflating the balloon, and retracting the hollow member within the positioning member. In an additional exemplary embodiment, a method for dilation of a stricture in a tubular organ or other hollow area of the human body comprises the steps of: positioning a balloon device (e.g., 600) within a stricture, extending the hollow member from the distal end of the positioning member through the stricture, inflating the balloon with a fluid, contacting the stricture with the balloon by retracting the hollow member, dilating the stricture, and retracting the hollow member within the positioning member.

Also disclosed herein are inventions relating to methods for resurfacing mucosa of tubular organs. For example, a method for resurfacing mucosa of tubular organs comprises the steps of positioning a balloon device (e.g., 100) within a tubular organ, inflating a balloon with a fluid of a certain temperature, making contact with the tubular organ for a certain period of time, deflating the balloon, and retracting the balloon device from the tubular organ. In certain embodiments, the method for resurfacing mucosa of tubular organs can further comprise rotating, extending, or retracting the balloon one or more times while inflated.

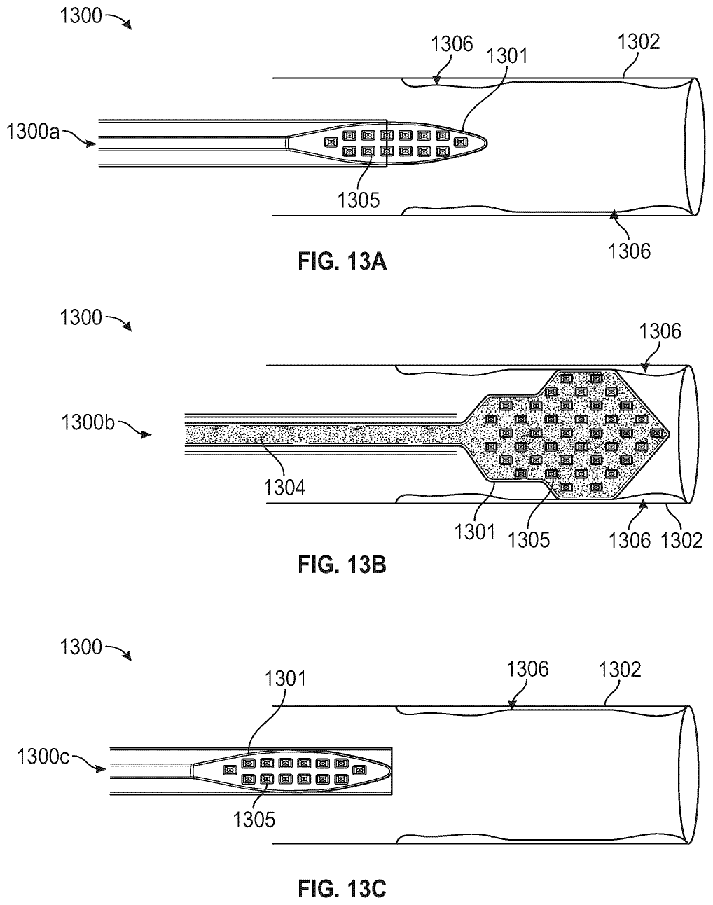

Turning now to FIGS. 13A-C, an embodiment of a method for resurfacing mucosa of tubular organs is illustrated. In balloon device 1300, balloon 1301 is shown having a textured surface 1305 (here, raised shapes). States 1300a and 1300b, respectively, illustrate the progression of balloon device 1300 relative to tubular organ 1302 as balloon device 1300 is positioned at target area 1306 of tubular organ 1302 in need of resurfacing and then balloon 1301 extends out from balloon device 1300 and is inflated with a fluid of a certain temperature 1304. In certain embodiments of the invention, fluid 1304 is heated or cooled. For example, in certain embodiments, fluid 1304 is heated water. In state 1300b, target area 1306 is thermally treated (hot or cold) when the surface of balloon 1301 contacts the target area. Because balloon 1301 in this embodiment features a textured surface 1305 having varying heights, balloon 1301 may be applied to target tissue with pressure so that the textured surface reaches varying depths of the target tissue depending on the relative heights of the textured surface 1305 of balloon 1301. In this way, the thermal treatment may be selectively administered depending on the configuration of the textured surface of balloon 1301. States 1300b and 1300c, respectively, illustrate the progression of balloon device 1300 during treatment relative to tubular organ 1302 as balloon 1301 is deflated and then removed from target tissue 1306. In state 1300c, thermal treatment has been administered to target area 1306 in a depth-selective manner in order to create healthy growth of the mucosa.

In an exemplary embodiment, a method for mucosal resurfacing or ablation comprises the steps of: positioning a balloon device (e.g., 600) at a target tissue, heating or cooling a fluid to a temperature above or below room temperature, inflating the balloon with the fluid, contacting a mucosal surface with the balloon, deflating the balloon, and retracting the balloon into the balloon device.

Also disclosed herein are methods for using balloon devices in conjunction with one or more guidewires. For example, a method for using a balloon device in conjunction with one or more guidewires comprises the steps of positioning a guidewire within a tubular organ inserting the proximal end of the guidewire into the distal end of the guidewire channel of a balloon device (e.g., balloon device 600 of FIGS. 6A-C), and advancing the balloon device distally along the guidewire. The method for using a balloon device in conjunction with one or more guidewires can further comprise positioning the balloon device within the tubular organ In preferred embodiments, the method for using a balloon device in conjunction with one or more guidewires can further comprise deploying the balloon device, wherein deploying the balloon device can comprise any of the other methods disclosed herein. In further embodiments, the method for using a balloon device in conjunction with one or more guidewires can further comprise retracting the guidewire and replacing it with the same guidewire or with one or more different guidewires one or more times while leaving the balloon device positioned within a tubular organ. In further embodiments, the method for using a balloon device in conjunction with one or more guidewires can further comprise retracting the balloon device and replacing it with the same balloon device or with one or more different balloon devices one or more times while leaving the guidewire positioned within a tubular organ. The one or more guidewires and the one or more balloon devices can be positioned through an endoscope or directly through a patient stoma or orifice.

In addition to tubular organs, certain embodiments of the invention may be used in other hollow areas of the body.

What is claimed is:

1. A tissue biopsy balloon device, comprising:
a balloon that when inflated increases in diameter step-wise along at least a longitudinal axis from its proximal end to a region of maximum diameter and decreases in diameter step-wise along at least its longitudinal axis from the region of maximum diameter to its distal end and comprises a textured surface;
a hollow member operable to extend, position, and retract the balloon and to inject and remove a fluid to inflate or deflate the balloon; and
a positioning member operable to position the hollow member and the balloon.

2. The balloon device of claim 1, wherein the balloon comprises one or more materials from the group consisting of silicone, latex, polyester, nylon, polyurethane and medical grade plastic.

3. The balloon device of claim 1, wherein the hollow member comprises one or more materials from the group consisting of silicone, latex, polyurethane, and medical grade plastic.

4. The balloon device of claim 1, wherein the positioning member comprises one or more materials from the group consisting of silicone, latex, polyurethane, and medical grade plastic.

5. The balloon device of claim 1, wherein the positioning member comprises one or more items from the group consisting of catheter, endoscope, lumen, sheath, forceps, and snare.

6. The balloon device of claim 1, wherein the fluid comprises one or more items from the group consisting of sterile water, saline, liquid nitrogen, contrast, radiopaque fluid, carbon dioxide, and oxygen.

7. The balloon device of claim 1, wherein the balloon when inflated is in the range of about 2.0-300 mm in length with a maximum diameter between 2.0-30.0 mm.

8. The balloon device of claim 1, wherein the balloon, when inflated, comprises:
a proximal first segment that is about 0.5-29.5 mm in length with a distally increasing diameter and wherein the proximal first segment forms an angle of about 30-45 degrees outwards from the longitudinal axis of the balloon;
a second segment, located distal to the proximal first segment, that is about 0.5-297.7 mm in length with a diameter of about 0.5-29.5 mm;
a third segment, located distal to the second segment, that is about 0.5-29.5 mm in length with a distally increasing diameter and wherein the third segment forms an angle of about 30-45 degrees outwards from the longitudinal axis of the balloon;
a fourth segment, located distal to the third segment, that is about 0.5-297.7 mm in length with a diameter of about 2.0-30.0 mm; and
a distal fifth segment that is about 1.4-30.0 mm in length with a distally decreasing diameter and wherein the distal fifth segment forms an angle of about 30-45 degrees inwards towards the longitudinal axis of the balloon.

9. The balloon device of claim 1, wherein the balloon, when inflated, comprises:
a proximal first segment that is about 1.4-30.0 mm in length with a distally increasing diameter and wherein the proximal first segment forms an angle of about 30-45 degrees outwards from the longitudinal axis of the balloon; and
a distal second segment, located distal to the proximal first segment, that is about 1.4-30.0 mm in length with a distally decreasing diameter and wherein the second segment forms an angle of about 30-45 degrees inwards towards the longitudinal axis of the balloon.

10. The balloon device of claim 1, wherein the balloon, when inflated, comprises:
a proximal first segment that is about 0.5-297.0 mm in length with a diameter of about 0.5-29.5 mm;
a second segment, located distal to the proximal first segment, that is about 1.0-29.5 mm in length with a distally increasing diameter and wherein the second segment forms an angle of about 30-45 degrees outwards from the longitudinal axis of the balloon;
a third segment, located distal to the second segment, that is about 0.5-297.0 mm in length with a diameter of about 2.0-30.0 mm;
a fourth segment, located distal to the third segment, that is about 1.0-29.5 mm in length with a distally decreasing diameter and wherein the fourth segment forms an angle of about 30-45 degrees inwards towards the longitudinal axis of the balloon; and
a distal fifth segment that is about 0.5-297.0 mm in length with a diameter of about 0.5-29.5 mm.

11. The balloon device of claim 1, wherein the balloon, when inflated, comprises:
a proximal first segment that is about 0.5-28.0 mm in length with a distally increasing diameter and wherein the proximal first segment forms an angle of about 30-45 degrees outwards from the longitudinal axis of the balloon;
a second segment, located distal to the proximal first segment, that is about 0.5-296.2 mm in length with a diameter of about 0.5-28.0 mm;
a third segment, located distal to the second segment, that is about 0.5-28.0 mm in length with a distally increasing diameter and wherein the third segment forms an angle of about 30-45 degrees outwards from the longitudinal axis of the balloon;
a fourth segment, located distal to the third segment, that is about 0.5-296.2 mm in length with a diameter of about 1.0-28.5 mm;
a fifth segment that is about 0.5-28.0 mm in length with a distally increasing diameter and wherein the fifth segment forms an angle of about 30-45 degrees outwards from the longitudinal axis of the balloon;
a sixth segment, located distal to the fifth segment, that is about 0.5-296.2 mm in length with a diameter of about 1.5-29.0 mm;
a seventh segment, located distal to the sixth segment, that is about 0.5-28.0 mm in length with a distally increasing diameter and wherein the seventh segment forms an angle of about 30-45 degrees outwards from the longitudinal axis of the balloon;
an eighth segment, located distal to the seventh segment, that is about 0.5-296.2 mm in length with a diameter of about 2.0-29.5 mm;
a ninth segment that is about 0.5-28.0 mm in length with a distally increasing diameter and wherein the ninth segment forms an angle of about 30-45 degrees outwards from the longitudinal axis of the balloon;
a tenth segment, located distal to the ninth segment, that is about 0.5-296.2 mm in length with a diameter of about 2.5-30.0 mm;
an eleventh segment, located distal to the tenth segment, that is about 1.7-29.5 mm in length with a distally decreasing diameter and wherein the eleventh segment forms an angle of about 30-45 degrees inwards towards the longitudinal axis of the balloon; and
a distal twelfth segment, located distal to the eleventh segment, that is about 0.5-296.2 mm in length with a diameter of about 0.5-29.5 mm.

12. The balloon device of claim 1, wherein the balloon, when inflated, comprises:
a proximal first segment that is about 0.5-294.8 mm in length with a diameter of about 0.5-28.5 mm;
a second segment, located distal to the proximal first segment, that is about 0.5-28.0 nm in length with a distally increasing diameter and wherein the second segment forms an angle of about 30-45 degrees outwards from the longitudinal axis of the balloon;
a third segment, located distal to the second segment, that is about 0.5-294.8 mm in length with a diameter of about 1.0-29.0 mm;
a fourth segment that is about 0.5-28.0 mm in length with a distally increasing diameter and wherein the fourth segment forms an angle of about 30-45 degrees outwards from the longitudinal axis of the balloon;
a fifth segment, located distal to the fourth segment, that is about 0.5-294.8 mm in length with a diameter of about 1.5-29.5 mm;
a sixth segment, located distal to the fifth segment, that is about 0.5-28.0 mm in length with a distally increasing diameter and wherein the sixth segment forms an angle of about 30-45 degrees outwards from the longitudinal axis of the balloon;
a seventh segment, located distal to the sixth segment, that is about 0.5-294.8 mm in length with a diameter of about 2.0-30.0 mm;
an eighth segment that is about 0.5-28.0 mm in length with a distally decreasing diameter and wherein the eighth segment forms an angle of about 30-45 degrees inwards towards the longitudinal axis of the balloon;
a ninth segment, located distal to the eighth segment, that is about 0.5-294.8 mm in length with a diameter of about 1.5-29.5 mm;
a tenth segment, located distal to the ninth segment, that is about 0.5-28.0 mm in length with a distally decreasing diameter and wherein the tenth segment forms an angle of about 30-45 degrees inwards towards the longitudinal axis of the balloon;
an eleventh segment, located distal to the tenth segment, that is about 0.5-294.8 mm in length with a diameter of about 1.0-29.0 mm;
a twelfth segment, located distal to the eleventh segment, that is about 0.5-280 mm in length with a distally decreasing diameter and wherein the twelfth segment forms an angle of about 30-45 degrees inwards towards the longitudinal axis of the balloon; and
a distal thirteenth segment, located distal to the twelfth segment, that is about 0.5-294.8 mm in length with a diameter of about 0.5-28.5 mm.

13. The balloon device of claim 1, wherein the balloon, when inflated, comprises:
a proximal first segment that is about 0.5-29.0 mm in length with a distally increasing diameter and wherein the proximal first segment forms an angle of about 30-45 degrees outwards from the longitudinal axis of the balloon;
a second segment, located distal to the proximal first segment, that is about 0.5-297.9 mm in length with a diameter of about 0.5-29.0 mm;
a third segment, located distal to the second segment, that is about 0.5-29.0 mm in length with a distally increasing diameter and wherein the third segment forms an angle of about 30-45 degrees outwards from the longitudinal axis of the balloon,
a fourth segment, located distal to the third segment, that is about 0.5-297.9 mm in length with a diameter of about 1.0-29.5 mm;
a fifth segment, located distal to the fourth segment, that is about 0.5-29.0 mm in length with a distally increasing diameter and wherein the fifth segment forms an angle of about 30-45 degrees outwards from the longitudinal axis of the balloon;
a sixth segment, located distal to the fifth segment, that is about 0.5-297.9 mm in length with a diameter of about 2.0-30.0 mm; and
a distal seventh segment, located distal to the sixth segment, that is about 1.4-30.0 mm in length with a distally decreasing diameter and wherein the distal seventh segment forms an angle of about 30-45 degrees inwards towards the longitudinal axis of the balloon.

14. The balloon device of claim 1, wherein the balloon, when inflated, comprises:
a proximal first segment that is about 0.5-296.5 mm in length with a diameter of about 0.5-29.0 mm;
a second segment, located distal to the proximal first segment, that is about 0.5-28.5 mm in length with a distally increasing diameter and wherein the second segment forms an angle of about 30-45 degrees outwards from the longitudinal axis of the balloon;
a third segment, located distal to the second segment, that is about 0.5-296.5 mm in length with a diameter of about 1.0-29.5 mm;
a fourth segment, located distal to the third segment, that is about 0.5-28.5 mm in length with a distally increasing diameter and wherein the fourth segment forms an angle of about 30-45 degrees outwards from the longitudinal axis of the balloon;
a fifth segment, located distal to the fourth segment, that is about 0.5-296.5 mm in length with a diameter of about 2.0-30.0 mm;
a sixth segment, located distal to the fifth segment, that is about 0.5-28.5 mm in length with a distally decreasing diameter and wherein the sixth segment forms an angle of about 30-45 degrees inwards towards the longitudinal axis of the balloon;
a seventh segment, located distal to the sixth segment, that is about 0.5-296.5 mm in length with a diameter of about 7.0-29.5 mm;
an eighth segment, located distal to the seventh segment, that is about 05-28.5 mm in length with a distally decreasing diameter and wherein the eighth segment forms an angle of about 30-45 degrees inwards towards the longitudinal axis of the balloon; and
a distal ninth segment, located distal to the eighth segment, that is about 0.5-296.5 mm in length with a diameter of about 0.5-29.0 mm.

15. The balloon device of claim 1, wherein balloon device further comprises a sheath encasing the balloon, wherein the sheath has an inner diameter in the range of about 0.25-4.0 mm and an outer diameter in the range of about 05-5.0 mm.

16. The balloon device of claim 1, wherein the balloon device further comprises two stoppers comprising a first stopper positioned proximally to the balloon and a second stopper positioned distally to the balloon.

17. The balloon device of claim 16, wherein the stoppers comprise pyramids.

18. The balloon device of claim 1, wherein the textured surface comprises at least one clinical agent.

19. The balloon device of claim 1, wherein the textured surface comprises raised abrasive nubs.

20. The balloon device of claim 1, wherein the textured surface comprises one or more sample collection wells.

21. The balloon device of claim 1, wherein the balloon device comprises one or more silk threads affixed to the balloon along the longitudinal axis.

22. The balloon device of claim 1, wherein the balloon device comprises a guidewire channel through which one or more guidewires can be extended.

23. A balloon device, comprising:
A balloon comprising a textured surface;
a sheath;
a positioning member operable to position the balloon;
a hollow member having a longitudinal axis disposed at least partially within the positioning member;
a balloon inflation channel operable to inject and remove a fluid to inflate or deflate the balloon and; and
two spheres comprising a first sphere positioned proximally to the balloon along the hollow member's longitudinal axis and a second sphere positioned distally to the balloon along the hollow member's longitudinal axis.

24. The balloon device of claim 23, wherein the balloon, when inflated, increases in diameter along at least a longitudinal axis from a proximal end to a region of maximum diameter, and decreases in diameter along the longitudinal axis from the region of maximum diameter to a distal end.

25. A balloon device, comprising:
a balloon;
a positioning member operable to position the balloon, further comprising:
one or more backstops; and
a plug operable to provide a proximal seal to contain fluid within the positioning member;
a hollow member disposed within at least partially within the positioning member;
a guidewire channel;
an inflation channel comprising a fluid port at its proximal end and one or more inflation ports at its distal end, operable to inject and remove a fluid to inflate or deflate the balloon; and
a first and second stopper disposed on either side of the balloon.

26. The balloon device of claim 25, wherein the balloon comprises one or more materials from the group consisting of silicone, latex, polyester, nylon, polyurethane and medical grade plastic.

27. The balloon device of claim 25, wherein the positioning member comprises one or more materials from the group consisting of silicone, latex, polyurethane, and medical grade plastic.

28. The balloon device of claim 25, wherein the hollow member comprises one or more materials from the group consisting of silicone, latex, polyurethane, and medical grade plastic.

29. A method for collecting tissue samples from different depths at a target location comprising the steps of:
positioning a balloon device at a target location, wherein the balloon device comprises:
a balloon comprising a textured surface;
a positioning member;
a hollow member having a longitudinal axis;
a proximal stopper positioned proximally to the balloon along the hollow member's longitudinal axis;
a distal stopper positioned distally to the balloon along the hollow member's longitudinal axis;
inflating the balloon with a fluid;
contacting a tissue with the balloon;
deflating the balloon;
retracting the balloon within the positioning member; and
harvesting collected tissue from the balloon.

30. The method of claim 29 wherein the balloon comprises a plurality of diameters.

31. The method of claim 29 wherein the balloon increases in diameter step-wise along a longitudinal axis from its proximal end to a region of maximum diameter near the midpoint of the longitudinal axis and decreases in diameter step-wise along, its longitudinal axis from the region of maximum diameter to its distal end.

32. The method of claim 29 wherein the balloon increases in diameter step-wise along a longitudinal axis from its proximal end to a region of maximum diameter near its distal end.

33. The method claim of claim 29 wherein the textured surface comprises raised surfaces having a plurality of heights.

34. The method of claim 29 further wherein the textured surface comprises indentations.

35. The method of claim 29 further wherein the textured surface comprises depressions.

36. The method of claim 29 further wherein the textured surface comprises depressions and indentations.

37. The method of claim 29 further comprising the step of dragging the inflated balloon along the target tissue while the balloon is in contact with the tissue.

38. A method of clinical or diagnostic agent administration comprising the steps of:
positioning a balloon device at a target tissue, wherein the balloon device comprises:
a balloon comprising a textured surface and a clinical or diagnostic agent coating;
a positioning member;
a hollow member having a longitudinal axis;
a proximal stopper positioned proximally to the balloon along the hollow member's longitudinal axis;
a distal stopper positioned distally to the balloon along the hollow member's longitudinal axis;
inflating the balloon with a fluid;
contacting a tissue with the balloon;
applying the clinical or diagnostic agent to the target tissue;
deflating the balloon; and
retracting the balloon within the positioning member.

39. The method of claim 38 wherein the balloon comprises a plurality of diameters.

40. The method of claim 38 wherein the balloon increases in diameter step-wise along at least a longitudinal axis from its proximal end to a region of maximum diameter near the midpoint of the longitudinal axis and decreases in diameter step-wise along the longitudinal axis from the region of maximum diameter to its distal end.

41. The method of claim 38 wherein the balloon increases in diameter step-wise along a longitudinal axis from its proximal end to a region of maximum diameter near its distal end.

42. The method of the claim 38 wherein the textured surface comprises raised surfaces having a plurality of heights.

43. The method of claim 38 wherein the textured surface comprises indentations or depressions.

44. The method of claim 38 further comprising the step of dragging the inflated balloon along the target tissue while the balloon is in contact with the tissue.

45. A method for dilation of a stricture in a tubular organ or other hollow area of the human body comprising the steps of:
positioning a balloon device on either side of a stricture, wherein the balloon device comprises:
a balloon comprising a textured surface and a longitudinal axis, and a diameter that is at its maximum at a location near the midpoint of the longitudinal axis and that decreases step-wise from its midpoint to its proximal end and from its midpoint to its distal end;
a positioning member; and
a hollow member;
extending the hollow member from the distal end of the positioning member through the stricture;
inflating the balloon with a fluid;
contacting the stricture with the balloon by retracting the hollow member;
dilating the stricture;
deflating the balloon; and
retracting the hollow member within the positioning member.

46. A method for dilation of a stricture in a tubular organ or other hollow area of the human body comprising the steps of:
positioning a balloon device within a stricture, wherein the balloon device comprises:
a balloon comprising a textured surface and a longitudinal axis, and a diameter that increases step-wise along a longitudinal axis from its proximal end to a region of maximum diameter near its distal end;
a positioning member; and
a hollow member;
extending the hollow member from the distal end of the positioning member through the stricture;
inflating the balloon with a fluid;
contacting the stricture with the balloon by retracting the hollow member;
dilating the stricture;
deflating the balloon; and
retracting the hollow member within the positioning member.

47. A method for mucosal resurfacing or ablation comprising the steps of:
positioning a balloon device at a target tissue, wherein the balloon device comprises:
a balloon comprising a textured surface and a longitudinal axis, and a diameter that is at its maximum at a location near the midpoint of the longitudinal axis and that decreases step-wise from its midpoint to its proximal end and from its midpoint to its distal end;
a positioning member; and
a hollow member;
heating or cooling a fluid to a temperature above or below room temperature;
inflating the balloon with the fluid;
contacting a mucosal surface with the balloon;
deflating the balloon; and
retracting the balloon into the balloon device.

48. A method for mucosal resurfacing or ablation comprising the steps of:
positioning a balloon device at a target tissue, wherein the balloon device comprises:
a balloon comprising a textured surface and a longitudinal axis, and a diameter that increases step-wise along a longitudinal axis from its proximal end to a region of maximum diameter near its distal end;
a positioning member; and
a hollow member;
heating or cooling a fluid to a temperature above or below room temperature;
inflating the balloon with the fluid;
contacting a mucosal surface with the balloon;
deflating the balloon; and
retracting the balloon into the balloon device.

49. The method of claim 47, wherein the fluid comprises one or more items from the group consisting of sterile water, saline, liquid nitrogen, contrast, radiopaque fluid, and oxygen.

50. The method of claim 48, wherein the fluid comprises one or more items from the group consisting of sterile water, saline, liquid nitrogen, contrast, radiopaque fluid, and oxygen.

51. The method of claim 47 wherein the fluid is heated above room temperature.

52. The method of claim 47 wherein the fluid is cooled below room temperature.

53. The method of claim 48 wherein the fluid is heated above room temperature.

54. The method of claim 48 wherein the fluid is cooled below room temperature.

55. A method of positioning a balloon device a tissue location along a guidewire, comprising:
positioning a guidewire within a tubular organ;
inserting a proximal end of the guidewire into a distal end of the guidewire channel of a balloon device, wherein the balloon device comprises a balloon, a positioning member operable to position the balloon, one or more backstops, a plug operable to provide a proximal seal to contain fluid within the positioning member, a hollow member disposed at least partially within the positioning member, a guidewire channel, an inflation channel comprising a fluid port at its proximal end and one or more inflation ports at its distal end, operable to inject and remove a fluid to inflate or deflate the balloon, and a first and second stopper disposed on either side of the balloon;
advancing the balloon device distally along the guidewire; and
positioning the balloon device within the tubular organ.

56. The method of claim 55, further comprising:
retracting the guidewire; and
replacing the guidewire with a different guidewire while leaving the balloon device positioned within the tubular organ.
